(19) United States
(12) Patent Application Publication
(10) Pub. No.: US 2023/0031898 A1
(43) Pub. Date: Feb. 2, 2023

(54) METHOD OF CELL-FREE DNA ANALYSIS TO IDENTIFY HIGH-RISK METASTATIC PROSTATE CANCER

(71) Applicant: Washington University, St. Louis, MO (US)

(72) Inventors: Aadel CHAUDHURI, Chesterfield, MO (US); Christopher MAHER, Olivette, MO (US); Russell PACHYNSKI, St. Louis, MO (US)

(21) Appl. No.: 17/783,333

(22) PCT Filed: Dec. 10, 2020
(86) PCT No.: PCT/US2020/064321

§ 371 Date: Jun. 8, 2022

Related U.S. Application Data
(60) Provisional application No. 62/946,021, filed on Dec. 10, 2019.
(60) Provisional application No. 62/946,660, filed on Dec. 11, 2019.

(51) Int. Cl. C12Q 1/6886; C12Q 1/6827

(52) U.S. Cl. CPC C12Q 1/6886; C12Q 1/6827; C12Q 2600/106; C12Q 2600/156; C12Q 2600/118; C12Q 2600/112

(57) ABSTRACT

Disclosed here in are methods and kits for identifying a prostate cancer treatment for a subject. The methods include obtaining a fluid sample from the subject, the fluid sample comprising noncellular DNA (ncDNA) from the subject, transforming the ncDNA into a plurality of genomic variations to determine if the ncDNA contains castration-resistant structural variations including at least one of a genomic alteration in AR encoding an androgen receptor and a genomic alteration of an AR enhancer; and identifying the prostate cancer treatment for the subject based on the plurality of genomic variations.

### CROSS-REFERENCE TO RELATED APPLICATIONS

The present application claims priority to U.S. Provisional Patent Application No. 62/946,660, filed Dec. 11, 2019, and U.S. Provisional Patent Application No. 62/946,021, filed Dec. 10, 2019, the entire contents and disclosures of which are incorporated herein by reference.

### STATEMENT REGARDING FEDERALLY SPONSORED RESEARCH OR DEVELOPMENT

Not applicable.

### TECHNICAL FIELD

The present disclosure generally relates to methods of diagnosing and treating prostate cancer. More specifically, the present disclosure relates to methods of diagnosing and treating high-risk metastatic prostate cancer using a circulating tumor DNA (ctDNA) assay.

### BACKGROUND

Prostate cancer is the most common non-skin cancer among men in the United States, with an estimated ˜175,000 new cases diagnosed this year. The most deadly form of prostate cancer, metastatic castration-resistant prostate cancer (mCRPC), is treated commonly first-line with the androgen receptor (AR)-directed therapies Abiraterone and Enzalutamide, and these are recommended and approved agents for this indication. However, patients can become resistant to these targeted therapies and unfortunately these patients with high-risk mCRPC have a very poor prognosis with a median survival of only ˜5.5 months. Recent evidence shows that ˜10-20% of these AR-refractory mCRPC patients express AR-V7, which led to the development of an assay for detecting AR-V7 in circulating tumor cells (CTCs) which now constitutes the clinical standard-of-care. However, the sensitivity of this assay is very poor, with ˜80-90% of assessed patients scored as false negatives, and the sensitivity drops off even further (˜3%) when tested prior to first-line mCRPC treatment. There is thus an imminent need for a more sensitive assay to detect AR-refractory mCRPC sooner.

With an increasing number of effective treatments for metastatic prostate cancer (PCa) (see FIG. 6), there is an urgent need for molecular biomarkers that allow improved sequencing of therapies and selection of agents for distinct molecular sub-types. Whole-exome and transcriptome sequencing studies of primary prostate tumors and metastatic tumor biopsies have given insights into the complexity and distribution of genome-wide aberrations. However, obtaining biopsies containing sufficient tumor from CRPC patients can be challenging, especially from men with low-volume disease who represent the majority of men relapsing with mCRPC. Moreover, repeated sampling to obtain real-time molecular information as patients develop treatment-resistant disease is often impractical. The recently clinically validated circulating tumor cell (CTC)-based assay for detecting an aberrant AR splice variant (AR-V7) as a treatment selection biomarker highlights the potential value of liquid biopsy based analysis in mCRPC patients. However, the sensitivity of this test for identifying AR-refractory mCRPC remains low at only ˜10-20% in tested patients, and only ˜3% prior to first line treatment for mCRPC when it might prove clinically most useful. Assessment of cell-free DNA (cfDNA) has recently emerged as another noninvasive means to assess clinically relevant genomic alterations in multiple cancer types. Cell-free DNA assessment of circulating tumor DNA (ctDNA) has been shown to be highly sensitive for detecting tumor-specific genomic mutations with capability to detect post-treatment minimal residual disease (MRD).

Metastatic castration resistant prostate cancer (mCRPC) is the deadliest form of prostate cancer. Outcomes have improved significantly with the advent of targeted AR-directed therapies such as abiraterone and enzalutamide. However, these targeted therapies are sometimes accompanied by the development of resistance to the therapy. A variety of therapy-induced resistance mechanisms are summarized in FIG. 7. For example, 20-40% of patients exhibit primary resistance to these targeted therapeutics, and have significantly worse survival. These high-risk prostate cancer patients have been shown to succumb rapidly to their disease with a median survival of only ˜6 months. Other patients develop secondary resistance to AR-directed therapy, responding well initially before eventually developing resistance. There is thus an urgent need for molecular biomarkers that can identify resistance to AR-directed therapy early, which would enable clinicians to consider alternate treatments (i.e. chemotherapy or immunotherapy) instead.

The recently clinically validated circulating tumor cell (CTC)-based assay for detecting an aberrant AR splice variant (AR-V7) as a treatment selection biomarker highlights the potential value of liquid biopsy based analysis in mCRPC patients. However, the sensitivity of this test for identifying AR-refractory mCRPC remains low at only ˜10-20% in tested patients, and only ˜3% prior to first line treatment for mCRPC when it might prove clinically most useful. Thus, while shown to have reasonable specificity and approved for clinical use, there is clearly a need for more sensitive biomarkers to reliably detect primary resistance to AR-directed therapy early.

Assessment of cell-free DNA (cfDNA) has recently emerged as a noninvasive means to assess clinically relevant genomic alterations in multiple cancer types including prostate cancer. Cell-free DNA assessment of circulating tumor DNA (ctDNA) has been shown to be highly sensitive for detecting tumor-specific genomic mutations with capability to detect post-treatment minimal residual disease. In metastatic prostate cancer, detection sensitivities have been shown to be high prior to treatment initiation, and it has been shown that copy number alterations in the androgen receptor gene body can be reliably measured. Still, it remains to be seen if measuring these alterations, especially those involving AR, can robustly identify resistance to ARdirected therapy.

Other objects and features will be in part apparent and in part pointed out hereinafter.

### SUMMARY

Various aspects of the present disclosure relate to a method for identifying a prostate cancer treatment for a subject, the method including obtaining a fluid sample from the subject, the fluid sample including noncellular DNA (ncDNA); transforming the ncDNA into a plurality of genomic variations to determine if the ncDNA contains castration-resistant structural variations including at least one of an amplification of an AR encoding an androgen receptor and an amplification of a distal AR enhancer; and identifying the prostate cancer treatment for the subject based on the plurality of genomic variations.

In various aspects, the step of identifying the prostate cancer treatment for the subject based on the plurality of genomic variations includes: identifying a non-AR focused treatment if the plurality of genomic variations includes at least one of the castration-resistant structural variations; and identifying an AR-focused treatment if the plurality of genomic variations does not include at least one of the castration-resistant structural variations. In various aspects, the non-AR focused treatment includes one or more of immunotherapy, radiotherapy, targeted therapy, and chemotherapy; and the AR-focused treatment includes one or more AR-directed drugs, such as Abiraterone and Enzalutamide.

In various aspects, the method further includes determining if the plurality of genomic variations contains DNA-repair deficiency-related structural variations including at least one of CDK12 mutations with tandem duplications, TP53 inactivation with inverted rearrangements and chromothripsis, and BRCA2 inactivation with deletions; and identifying a DNA damage repair treatment if the plurality of genomic variations includes at least one of the DNA-repair deficiency-related structural variations. The DNA damage repair treatment may include administration of a poly (ADP-ribose) polymerase (PARP) inhibitor.

In various aspects, the fluid sample includes at least one of a blood sample, a plasma sample, a urine sample, and a saliva sample.

In various aspects, the transforming step includes contacting the fluid sample with one or more probes, each including a gene sequence selected from a gene panel, wherein the one or more probes are configured to capture the castration-resistant structural variations in the fluid sample. In various aspects, the gene panel may include copy number control genes and clonal hematopoiesis genes. The probes may further be labeled with radioactive or non-radioactive labels.

Various aspects of the present disclosure additionally relate to an assay kit including and assay including a plurality of probes wherein the plurality of probes are configured to transform a fluid sample comprising ncDNA into a plurality of genomic variations wherein the genomic variations comprise at least one of an amplification or structural variation of an AR encoding an androgen receptor and an amplification or structural variation in an AR enhancer.

In various aspects, each probe of the plurality of probes includes a gene sequence selected from a gene panel, wherein the plurality of probes is configured to capture the plurality of genomic variations in the fluid sample. In various aspects, the plurality of genomic variations further comprise additional copy number alterations, fusions, rearrangements, single nucleotide variants and insertions/deletions and combinations thereof.

Various aspects of the present disclosure further relate to a method of treating prostate cancer including obtaining a fluid sample from a subject, the fluid sample including noncellular DNA (ncDNA) from the subject, transforming the ncDNA into a plurality of genomic variations to determine if the ncDNA contains castration-resistant structural variations including at least one of an amplification or structural variation of an AR encoding an androgen receptor, and an amplification or structural variation of an AR enhancer; and administering a prostate cancer treatment to the subject based on the plurality of genomic variations.

### DESCRIPTION OF THE DRAWINGS

Those of skill in the art will understand that the drawings, described below, are for illustrative purposes only. The drawings are not intended to limit the scope of the present teachings in any way.

FIG. 1 is an image summarizing a landscape of somatic and structural alterations in metastatic castration-resistant prostate cancer (mCRPC), including mutation and alteration frequencies of key genes, as elucidated by deep whole genome sequencing;

FIG. 2A contains a summary of whole genome and transcriptome analysis including graphs showing DNA amplification frequency (top), tandem duplication frequency (upper middle), tandem duplication bounds (lower middle), and H3K27ac average read coverage (bottom) at the AR locus;

FIG. 2B is a box and whisker plot summarizing AR expression in the presence/absence of DNA amplification at AR or at the peak;

FIG. 2C is a histogram of AR copies for various samples from the peak of the DNA amplification frequency graph of FIG. 2A showing that samples with tandem duplications of the AR enhancer region but not the AR locus (labeled red in the histogram) more frequently had AR unamplified or amplified at low levels;

FIG. 3 is a schematic illustration of an exemplary targeted hybrid-capture panel design process and ctDNA analysis;

FIG. 4 is a non-synonymous mutation heat map summarizing mutations from a population of mCRPC patients; 16 of 20 patients had nonsynonymous SNVs/indels detected;

FIG. 5 is a graph summarizing a representative result of an EnhanceAR-Seq ctDNA assay for DNA amplification in accordance with one aspect of the disclosure, showing AR enhancer and gene body amplification in a patient concurrently showing a CTC ARV7 negative result; the patient progressed rapidly (PSA 100->500->1000) and subsequently died with a PSA >1000;

FIG. 6 is a schematic diagram illustrating various prostate cancer therapies targeting the androgen receptor (AR) pathway;

FIG. 7 is a schematic diagram illustrating various mechanisms of resistance to AR-directed prostate cancer therapies;

FIG. 8 is a genome map summarizing various somatic and structural genomic alterations among a population of mCRPC patients;

FIG. 9 is a schematic diagram illustrating potential means of monitoring clonal evolution and resistance to AR-directed therapy in patients with mCRPC;

FIG. 10 is a schematic diagram illustrating a strategy for developing a targeted sequencing assay of plasma cell-free DNA for monitoring clonal evolution and resistance to AR-directed therapy;

FIG. 11 is a genomic map showing AR intervals;

FIG. 12 is a genomic map showing the AR region;

FIG. 13 is a genomic map showing the TMPRSS2 region;

FIG. 14 is a graph summarizing in silico performance of a mCRPC ctDNA targeted panel;

FIG. 15 is a genomic map showing deletion hotspots for the ERG and TMPRSS2 regions;

FIG. 16 is a schematic diagram illustrating a method of selecting a treatment for a mCRPC patient based on results from a mCRPC ctDNA targeted panel in accordance with one embodiment of the disclosure;

FIG. 17 is a genomic alteration map summarizing genomic alteration patterns of AR enhancer and AR regions in a metastatic prostate cancer patient population;

FIG. 18 is a summary of genomic alteration patterns characterizing TMPRSS2-ERG fusion events;

FIG. 19 is an genomic alteration map summarizing patterns of TMPRSS2-ERG fusion events in a patent population in which 7/41 patients (17%) exhibited TMPRSS2-ERG fusions;

FIG. 20A is a co-mutation map summarizing mutations, genomic alterations, characteristics and test results within a patient population, with the shown mutations and genomic alterations identified in plasma cfDNA on a per-patient basis;

FIG. 20B is an enlargement of a portion of the co-mutation map of FIG. 20A (demarcated by a superimposed rectangle), showing the most common genomic alterations on a per-patient basis in the metastatic prostate cancer cohort;

FIG. 21A is a bar graph summarizing the proportion of patients developing AR resistance in patients exhibiting/not exhibiting AR/enhancer gain in cfDNA;

FIG. 21B is a bar graph summarizing the proportion of patients developing AR resistance in patients exhibiting/not exhibiting the AR-V7 splice-variant protein in CTCs;

FIG. 22A is a Kaplan-Meier graph comparing progression-free survival of patients exhibiting/not exhibiting AR/enhancer gain in cfDNA;

FIG. 22B is a Kaplan-Meier graph comparing progression-free survival of AR-V7 positive/negative patients;

FIG. 22C is a Kaplan-Meier graph comparing overall survival of patients exhibiting/not exhibiting AR/enhancer gain in cfDNA;

FIG. 22D is a Kaplan-Meier graph comparing overall survival of CTC AR-V7 positive/negative patients;

FIG. 23A is a co-mutation plot based on cfDNA analysis of patients with metastatic prostate cancer treated with AR-directed therapy, wherein each column represents data from a single patient and wherein rates of queried genomic alterations are depicted by the bar graphs to the right;

FIG. 23B illustrates a proportion of patients with AR/enhancer genomically altered or wild type in cfDNA, who developed resistance or not to AR-directed therapy;

FIG. 24A is a heat map of all somatic SNVs detected in cell-free DNA from a cohort of patients, wherein resistance to AR-directed therapy is indicated below the bar graph as resistant on the left versus sensitive on the right;

FIG. 24B is a comparison of the number of SNVs detected in plasma cfDNA in the cohort of patients;

FIG. 24C illustrates ctDNA levels in AR-resistant versus AR-sensitive patients;

FIG. 25 is a comparison of AR gene body alterations detected by tumor and plasma cell-free DNA sequencing;

FIG. 26 illustrates AR-V7 detection in circulating tumor cells and its association with resistance to AR-directed therapy;

FIG. 27A illustrates progression-free survival (PFS) according to androgen receptor (AR)/enhancer alteration status in cell-free DNA (cfDNA) in a 40-patient prostate cancer cohort;

FIG. 27B illustrates overall survival (OS) according to androgen receptor (AR)/enhancer alteration status in cell-free DNA (cfDNA) in the 40-patient prostate cancer cohort;

FIG. 27C illustrates PFS according to androgen receptor (AR)/enhancer alteration status in cfDNA after excluding patients with secondary resistance to AR-directed therapy;

FIG. 27D illustrates OS according to androgen receptor (AR)/enhancer alteration status in cfDNA after excluding patients with secondary resistance to AR-directed therapy;

FIG. 28A illustrates PFS according to AR enhancer status in cfDNA;

FIG. 28B illustrates OS according to AR enhancer status in cfDNA;

FIG. 29A illustrates a serial time point liquid biopsy analysis for a patient PB078 on androgen receptor (AR) directed treatment;

FIG. 29B illustrates a serial time point liquid biopsy analysis for a patient PB087 on androgen receptor (AR) directed treatment;

FIG. 29C illustrates a serial time point liquid biopsy analysis for a patient PB203 on androgen receptor (AR) directed treatment;

FIG. 29D illustrates a serial time point liquid biopsy analysis for a patient PB140 on androgen receptor (AR) directed treatment;

FIG. 30 illustrates that cfDNA-detected alterations in a full AR locus including an AR enhancer was highly significant for inferior OS;

FIG. 31A illustrates that alterations of the AR/enhancer locus detected by an embodiment of the pipeline of the disclosure were strongly associated with patient PFS in mCRPC;

FIG. 31B illustrates that alterations of the AR/enhancer locus detected by an embodiment of the pipeline of the disclosure were strongly associated with patient OS in mCRPC; and

FIG. 31C illustrates that alterations of the AR/enhancer locus detected by an embodiment of the pipeline of the disclosure were strongly associated with a high hazard ratio of patient OS in mCRPC.

There are shown in the drawings arrangements, which are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

The term “subject” as used herein may generally refer to a mammal, such as a human, that is need of therapy for prostate cancer. As used herein, the subject may be a patient, such as a prostate cancer patient.

The term “genomic variation” as used herein refers to variations in the genome of a species and may include microscopic and submicroscopic subtypes, such as, but not limited to, deletions, nucleotide variations, duplications, copy-number variants, insertions, inversions and translocations.

The term “probe” as used herein refers to a single-stranded sequence of DNA or RNA used to search for its complementary sequence in a sample genome. The probe may be placed into contact with a sample under conditions that allow the probe sequence to hybridize with the complementary sequence. The probe may be labeled with a radioactive or chemical tag that allows binding to be visualized.

The term “transforming” as used herein refers to the act of contacting a fluid sample including ncDNA with one or more probes having a gene sequence configured to search for and hybridize with a complementary sequence on the ncDNA.

The term “fluid sample” as used herein refers to a blood sample, a plasma sample, a urine sample or a saliva sample or other bodily fluid sample obtained from a subject.

Cell-free DNA and circulating tumor cells (CTCs) have emerged as powerful analytes for identifying resistance to therapy across a variety of cancer types. In metastatic castration resistant prostate cancer (mCRPC), detection of the androgen-receptor splice variant 7 (AR-V7) messenger RNA in CTCs was shown to be highly specific for identifying resistance to AR-directed therapy in patients with metastatic prostate cancer. However, the sensitivity of assays using this approach is poor. The use of assays that use cell-free DNA (cfDNA) to track structural variations and amplifications associated with the AR gene and its enhancer are potentially superior at identifying resistance to AR-directed therapy with much greater sensitivity than the standard-of-care CTC assay.

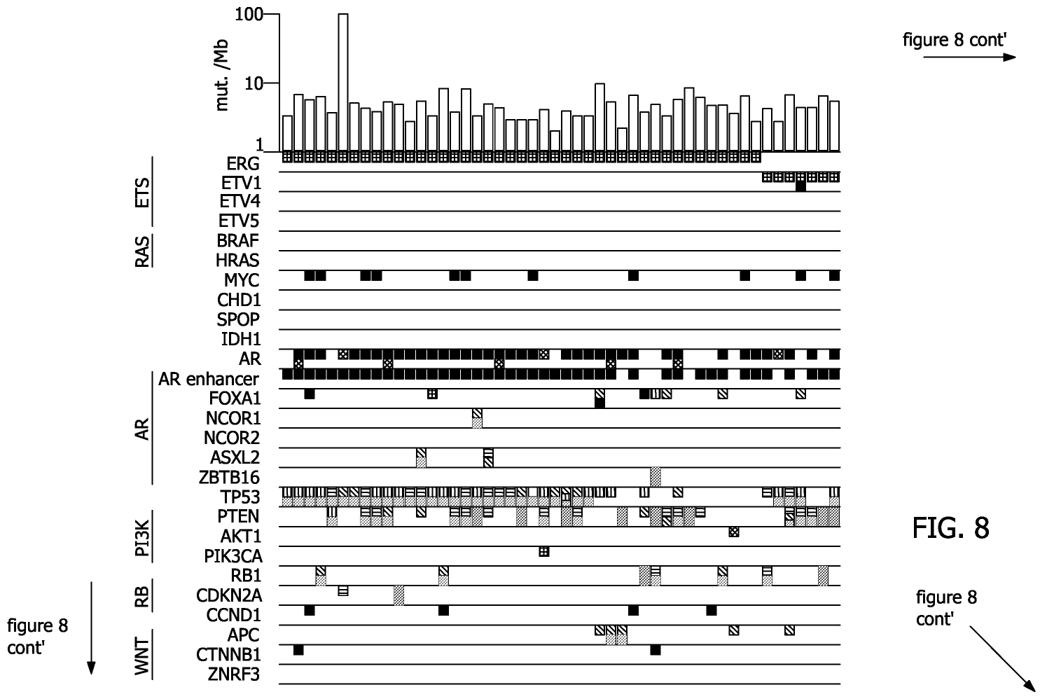

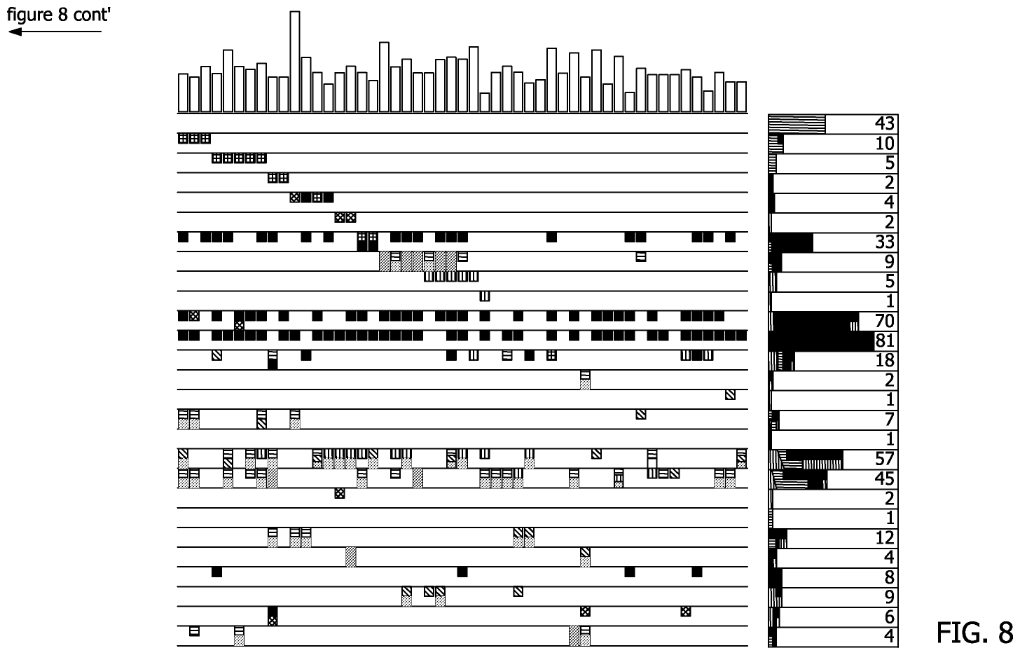

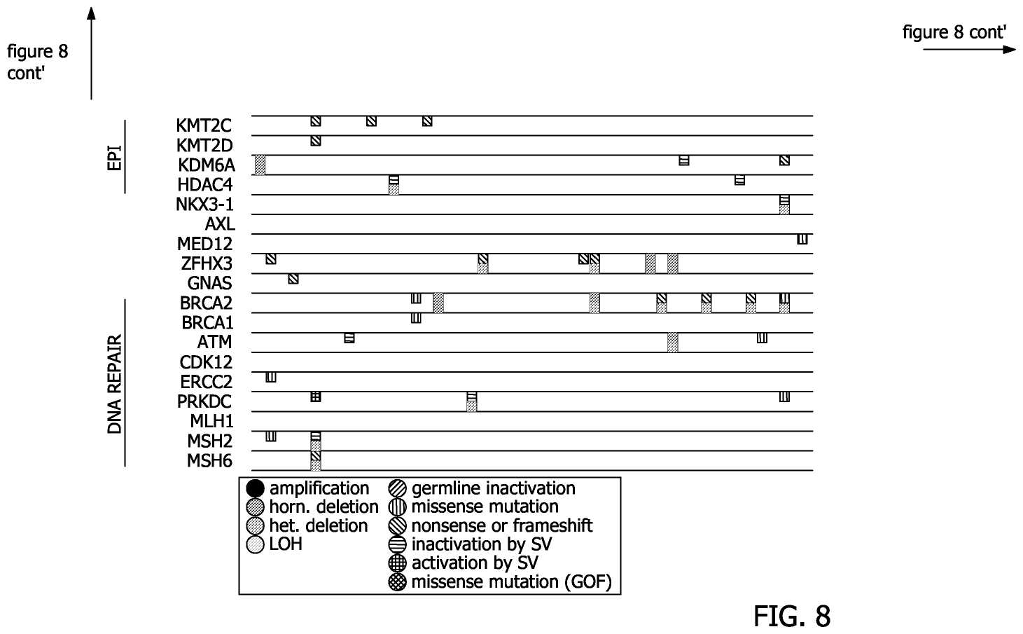

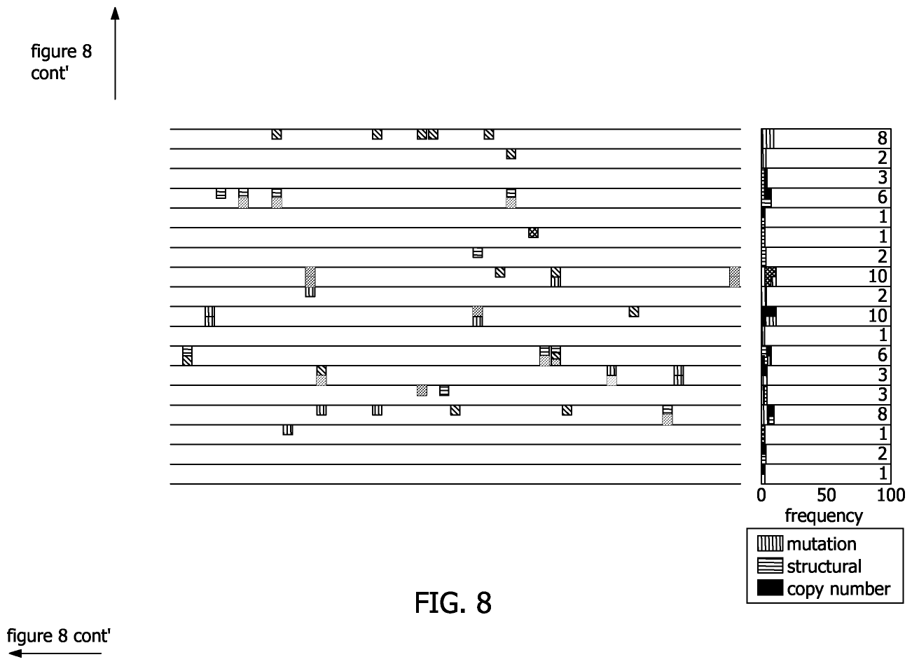

Integrative deep whole-genome and whole-transcriptome analyses of biopsies from metastatic castration-resistant prostate cancer (mCRPC) patients have been performed to discover the structural variants (SVs) underlying castration resistance. These analyses revealed stereotypic amplification of a distal enhancer region 624 kb upstream from AR in 81% of the mCRPC patients that correlated with significantly increased AR gene expression (see FIG. 8, overlaid blue rectangle). Tracking this genomic alteration thus represents an opportunity to detect, with greatly enhanced sensitivity, refractory mCRPC non-invasively. Non-invasive methods of tracking genomic alterations are desirable, because the serial biopsies typically used to monitor genomic alterations are not clinically practical for use in monitoring prostate tissue.

In one aspect, a method of monitoring genomic alterations associated with mCRPC non-invasively is disclosed. In this aspect, genomic alterations associated with mCRPC are monitored noninvasively in cfDNA using an assay configured to transform the cell-free DNA into a plurality of genomic variations to determine if the cfDNA contains castration-resistant alterations. In various aspects, the method enables detection and identification of high-risk metastatic prostate cancer using a biofluids-based liquid biopsy approach.

In some aspects, the disclosed method may enable a practitioner to identify an appropriate treatment for a patient. In one aspect, the disclosed method may enable the prediction of a patient's likelihood of developing resistance to androgen receptor (AR)-directed treatments, such as Abiraterone, Enzalutamide or Bicalutamide, earlier than is achievable using existing methods. In various aspects, the disclosed method may allow for identification of a treatment for mCRPC based on the presence of structural variants (SVs) underlying castration resistance in a fluid sample obtained from a subject. In some aspects, a non-AR focused treatment may be identified if structural variants (SVs) underlying castration resistance are present in the fluid sample. In some aspects an AR-focused treatment may be identified if structural variants (SVs) underlying castration resistance are not found in the fluid sample.

In various aspects, non-AR focused treatments may include immunotherapy (e.g., sipuleucel-T), radiotherapy (e.g., radium-223), targeted therapy (e.g., poly ADP-ribose polymerase (PARP) inhibitors) chemotherapy (e.g., docetaxel, cabazitaxel), and other prostate cancer treatments that do not act as androgen receptor inhibitors. In various aspects, AR-focused treatments may include pharmaceutical compositions such as abiraterone, enzalutamide and the like, that act as androgen receptor inhibitors or target the androgen/androgen receptor pathway.

In various aspects, the disclosed method may enable a practitioner to identify treatments for specific structural variants, such as DNA-repair deficiency-related structural variations and the like. Exemplary DNA-repair deficiency-related structural variations include CDK12 mutations with tandem duplications, TP53 inactivation with inverted rearrangements and chromothripsis, and BRCA2 inactivation with deletions and the like. Treatments may be selected from poly (ADP-ribose) polymerase (PARP) inhibitors and any other treatments effective for treating DNA-repair deficiency-related structural variations.

In various aspects, the method may include performing targeted hybrid-capture sequencing on patient samples, such as blood, plasma, or urine, using a customized panel that includes genes and genomic domains relevant to prostate cancer. In various aspects, the patient sample may be contacted with one or more probes, each including a gene sequence selected from the customized panel. The one or more probes may be configured to hybridize-capture castration-resistant structural variations in the patient sample. The resulting captured sequences may be analyzed to identify one or more parameters predictive of resistance to AR-directed treatment including, but not limited to, copy number alterations, single nucleotide variants, insertions/deletions, and genomic rearrangements. In one aspect, the resulting sequences may be analyzed to identify copy number alterations and rearrangements involving AR and its upstream enhancer, which are highly predictive for identifying resistance to AR-directed treatment.

In various aspects, the method may include isolating DNA from liquid biological samples, such as blood, plasma, or urine. In various aspects, the DNA isolated from liquid biological samples may include cell-free DNA (cfDNA) or noncellular DNA (ncDNA). The ncDNA from the sample may be subjected to an assay configured to capture structural variations that may indicate castration resistance. The captured structural variant sequences may then be subjected to a bioinformatics analysis.

In various aspects, the bioinformatics analysis may be customized. In various aspects, the bioinformatics analysis may be performed using any suitable bioinformatics tool to effectively identify one or more parameters predictive of resistance to AR-directed treatment including, but not limited to, copy number alterations, single nucleotide variants, insertions/deletions, and genomic rearrangements. In some aspects, the bioinformatics analysis may be performed using any suitable bioinformatics tool to effectively identify copy number alterations and rearrangements involving AR and its upstream enhancer, which are highly predictive for identifying resistance to AR-directed treatment

Without being bound by theory, the ability to track genomic events involving an AR enhancer robustly using a liquid biopsy technique may enable the identification of resistance to AR-directed treatment early, and may overcome a variety of shortcomings of existing methods. The disclosed method may identify resistance to AR-directed treatment in prostate cancer patients without the need for solid tissue biopsies. In various aspects, the disclosed method may allow for a noninvasive approach to identify high-risk metastatic prostate cancer patients early and select appropriate, personalized, therapy for such patients.

In addition, the disclosed method may detect tumor genomic events in plasma, thereby overcoming the shortcomings of existing invasive tumor-based methods, such as geographic tumor heterogeneity that introduces the risk of missing important tumor clones in the biopsy specimen, leading to an incorrect conclusion upon analysis. In various aspects, the disclosed method may allow for detection of tumor genomic events through biofluid (i.e. blood, urine) analysis, where geographic tumor heterogeneity is not likely to be an issue.

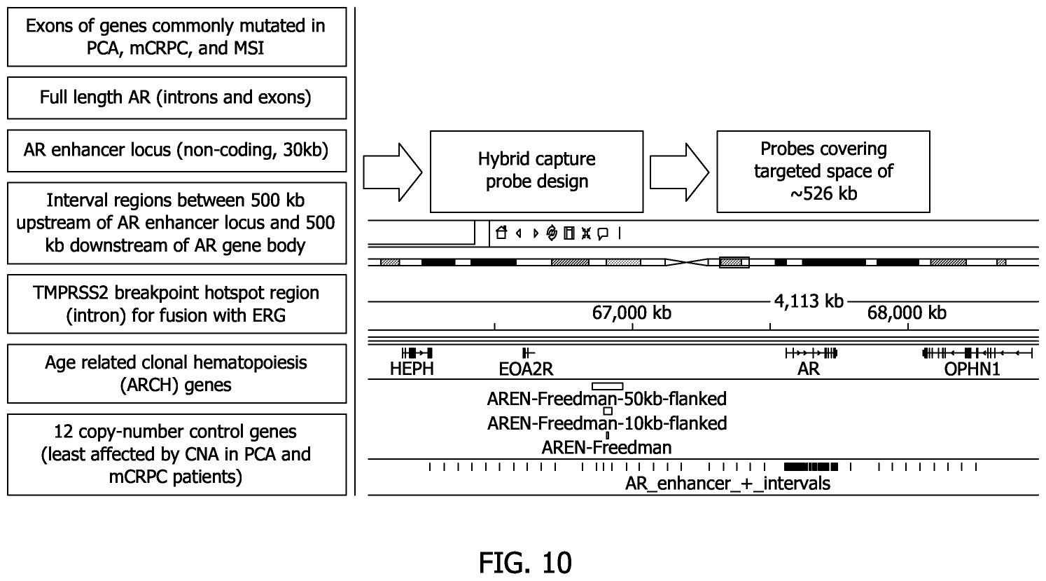

In various aspects, the disclosed method may be used to flexibly detect tumor genomic alterations including, but not limited to, tumor genomic alterations involving the Androgen Receptor gene and its regulatory elements, such as a distal enhancer, from nearly any biofluid, tissue, or cancer type. In some aspects, the disclosed method may enable simultaneous assessment of genomic alterations including copy number alterations in AR and its regulatory elements (including the distal enhancer), genomic rearrangements including the TMPRSS2:ERG fusion, and single nucleotide variations and insertions/deletions including those involving the TP53 gene. In some aspects, these analyses may be performed using a ˜526 kb panel involving ˜85 genes and genomic regions selected expressly for the purpose of tracking resistance to therapy in prostate cancer (see FIG. 10). In other aspects, the disclosed method may further enable identification of mutations in genes involved in microsatellite instability in prostate cancer patients, and may be useful for identifying patients who may benefit from various treatment strategies, including immune checkpoint blockade treatment strategies and the like.

In various other aspects, the disclosed method may further enable simultaneous tracking of genomic events relevant to a patient's solid tumor malignancy (mutations, fusions, copy number alterations) as well as clonal hematopoiesis (mutations in the genes DMT3A, TET2 and ASXL1). Using the disclosed method, genomic events related to the patient's primary malignancy may be delineated independently of potentially confounding clonal hematopoiesis mutations.

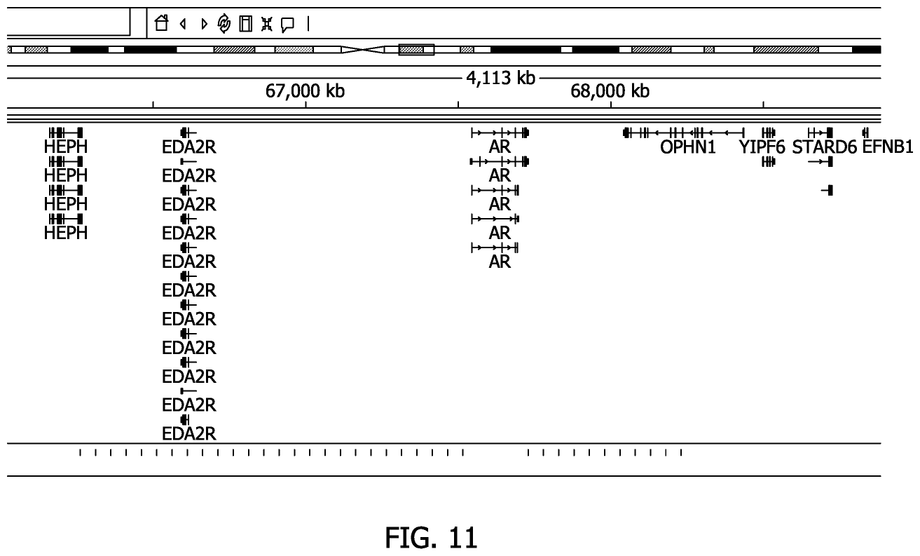

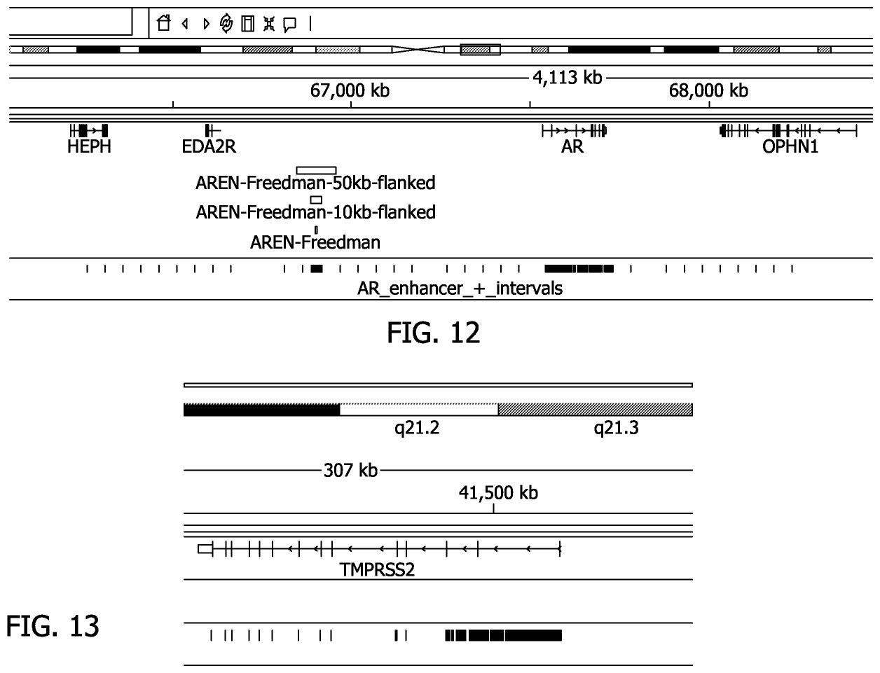

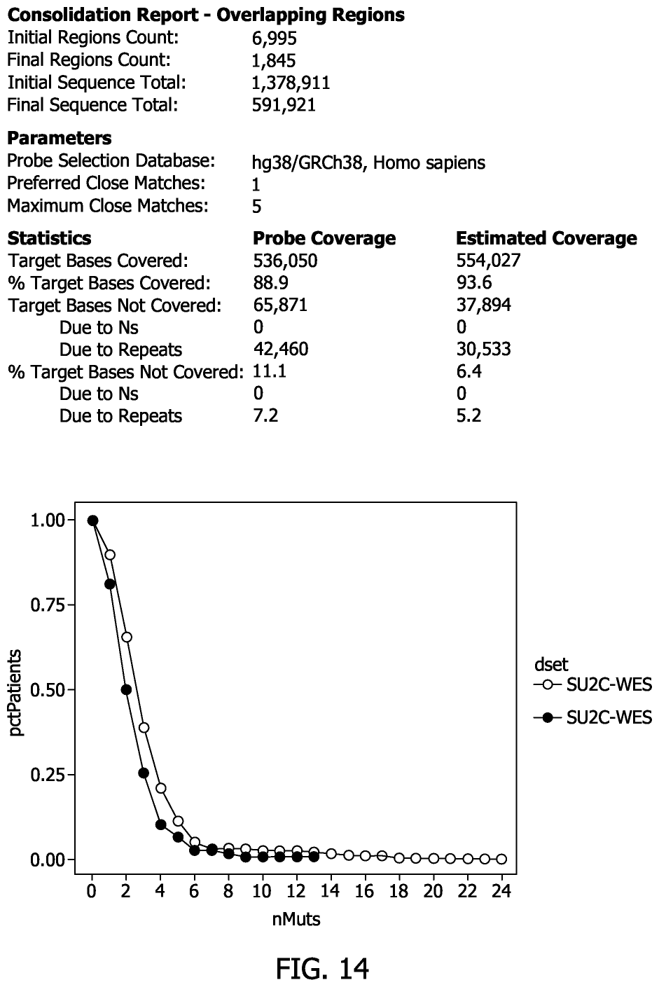

In one aspect, the customized gene panel may include the coding regions of 100 genes, summarized in Table 1 below. The 100 genes of the genomic panel of Table 1 include 12 copy number (CN) control genes, 3 genes related to control of clonal hematopoiesis of indeterminate potential (CHIP), and 85 genes associated with mCRPC and PCA (PRAD), including 6 genes associated with satellite instability (MSI). The mCRPC-associated genes include a full-length AR gene (including introns) as shown in FIGS. 11 and 12, an AR enhancer gene (9 kb with 10 kb flanking segments), intervals between the AR enhancer gene and the AR gene with 500 kb flanking segments, and a TMPRSS2 deletion hotspot (13 kb), as shown in FIG. 13. FIG. 14 is a summary of the performance of the genomic panel as evaluated in silico.

TABLE 1
Genes of mCRPC cfDNA Targeted Panel

| Gene | Description | Gene | Description | Gene | Description |
|---|---|---|---|---|---|
| ASXL1 | CHIP | CCND1 | PRAD | KDM6A | PRAD |
| DNMT3A | CHIP | CDK12 | PRAD | KMT2C | PRAD |
| TET2 | CHIP | CDK4 | PRAD | KMT2D | PRAD |
| ACTR1B | CN-control | CDK6 | PRAD | KRAS | PRAD |
| AKAP7 | CN-control | CDKN1B | PRAD | MDM2 | PRAD |
| ANKRD36 | CN-control | CDKN2A | PRAD | MDM4 | PRAD |
| APLN | CN-control | CHD1 | PRAD | MED12 | PRAD |
| CYP4F22 | CN-control | CLU | PRAD | MET | PRAD |
| CYP4F3 | CN-control | CTNNB1 | PRAD | MYC | PRAD |
| ELF4 | CN-control | CUL1 | PRAD | NCOA2 | PRAD |
| SLITRK2 | CN-control | ERCC1 | PRAD | NCOR1 | PRAD |
| SPANXN1 | CN-control | ERCC2 | PRAD | NCOR2 | PRAD |
| SPTY2D1 | CN-control | ERCC3 | PRAD | NFE2L2 | PRAD |
| TPTE | CN-control | ERCC4 | PRAD | NKX3-1 | PRAD |
| TRIM43 | CN-control | ERCC5 | PRAD | PIK3CA | PRAD |
| MLH1 | MSI | ERG | PRAD | PIK3CB | PRAD |
| MSH2 | MSI | ETV1 | PRAD | PIK3R1 | PRAD |
| MSH3 | MSI | ETV4 | PRAD | PRKDC | PRAD |
| MSH6 | MSI | ETV5 | PRAD | PTEN | PRAD |
| PMS1 | MSI | FANCA | PRAD | RAD51B | PRAD |
| PMS2 | MSI | FANCC | PRAD | RAD51C | PRAD |
| AKT1 | PRAD | FANCD2 | PRAD | RB1 | PRAD |
| AKT2 | PRAD | FANCE | PRAD | RNF43 | PRAD |
| AKT3 | PRAD | FANCF | PRAD | RUNX1 | PRAD |
| APC | PRAD | FANCG | PRAD | RYBP | PRAD |
| AR | PRAD | FBXW7 | PRAD | SMARCA1 | PRAD |
| ARID1A | PRAD | FOXA1 | PRAD | SPOP | PRAD |
| ASXL2 | PRAD | FOXP1 | PRAD | TMPRSS2 | PRAD |
| AIM | PRAD | GNAS | PRAD | TP53 | PRAD |
| ATR | PRAD | HDAC4 | PRAD | ZBTB16 | PRAD |
| AXL | PRAD | HRAS | PRAD | ZFHX3 | PRAD |
| BRAF | PRAD | HSD3B1 | PRAD | ZNRF3 | PRAD |
| BRCA1 | PRAD | IDH1 | PRAD | | |
| BRCA2 | PRAD | IDH2 | PRAD | | |

CHIP = clonal hematopoiesis of indeterminate potential control genes; CN-control = copy number control genes; MSI = microsatellite instability genes; PRAD = mCRPC/PCA genes

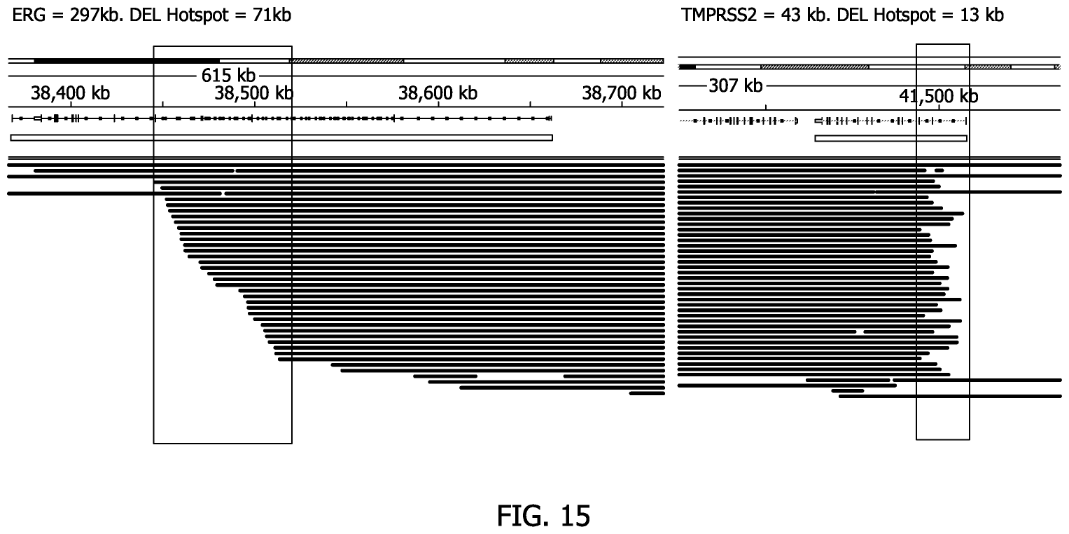

In other aspects, the genomic panel may be reduced in size by targeting key domains within larger genes, rather than the full length of the gene. By way of nonlimiting example, illustrated in FIG. 15, TMPRSS2-ERG fusion may be evaluated by targeting just the DEL hotspot within TMPRSS2 and/or the DEL hotspot in ERG, rather than targeting the full length sequences of these genes.

Although the disclosed method is described herein in relation to the analysis of blood plasma-derived cell-free DNA, the disclosed method may be suitable for the analysis of cell-free DNA derived from any bodily fluid including, but not limited to, urine, saliva, or any other suitable bodily fluid without limitation.

In various aspects, a method of treating metastatic prostate cancer is further disclosed. In various aspects, the method may include performing targeted hybrid-capture sequencing on patient samples, such as blood, plasma, or urine, using a customized panel that includes genes and genomic domains relevant to prostate cancer. In various aspects, the patient sample may be contacted with one or more probes, each including a gene sequence selected from the customized panel. The one or more probes may be configured to hybridize-capture castration-resistant structural variations in the patient sample. The resulting captured sequences may be analyzed to identify one or more parameters predictive of resistance to AR-directed treatment including, but not limited to, copy number alterations, single nucleotide variants, insertions/deletions, and genomic rearrangements. In one aspect, the resulting sequences may be analyzed to identify copy number alterations and rearrangements involving AR and its upstream enhancer, which are highly predictive for identifying resistance to AR-directed treatment, as disclosed below.

In some aspects, the disclosed method may enable a practitioner to administer an appropriate treatment for a patient. In one aspect, the disclosed method may enable the prediction of a patient's likelihood of developing resistance to androgen receptor (AR)-directed treatments, such as Abiraterone, Enzalutamide or Bicalutamide earlier than is achievable using existing methods. In various aspects, the disclosed method may allow for administration of a treatment for mCRPC based on the presence of structural variants (SVs) underlying castration resistance in a fluid sample obtained from a subject. In some aspects, a non-AR focused treatment may be administered if structural variants (SVs) underlying castration resistance are present in the fluid sample. In some aspects an AR-focused treatment may be administered if structural variants (SVs) underlying castration resistance are not found in the fluid sample.

In various aspects, the disclosed method may enable a practitioner to administer treatments for specific structural variants, such as DNA-repair deficiency-related structural variations and the like. Exemplary DNA-repair deficiency-related structural variations include CDK12 mutations with tandem duplications, TP53 inactivation with inverted rearrangements and chromothripsis, and BRCA2 inactivation with deletions and the like. Treatments may be selected from poly (ADP-ribose) polymerase (PARP) inhibitors and the like.

In various aspects, an assay kit that may be used to identify a metastatic prostate cancer treatment for a subject is disclosed. In some aspects, the kit may include a probe set including a plurality of probes. The probes may each include a gene sequence selected from a customized panel that includes genes and genomic domains relevant to prostate cancer. In various aspects the probes may be configured to hybridize-capture castration-resistant structural variations in a patient sample. In various aspects, the customized gene panel may be converted into a probe set using tools known to those of skill in the art. In other aspects, the kit may be configured in any way that enables transformation of a fluid sample comprising ncDNA into a plurality of genomic variations, such as castration-resistant structural variations. In various aspects, the assay kit may further include suitable tools known in the art to perform a bioinformatics analysis of the captured castration-resistant structural variations.

Although the systems and methods of the present disclosure are presented in the context of the detection of structural variations in patients with metastatic castration resistant prostate cancer (mCRPC), the systems and methods of the present disclosure are suitable for detecting structural variants and to select treatments in patients with earlier disease states (e.g., non-metastatic or metastatic hormone-sensitive prostate cancer) and other cancer types. By way of non-limiting example, the systems and methods of the present disclosure may be used to detect structural variants and to select treatments in non-metastatic very-high-risk hormone-sensitive patients being considered for Abiraterone (or other AR-targeted agents) vs. Docetaxel in addition to standard androgen deprivation therapy (ADT) after radiotherapy.

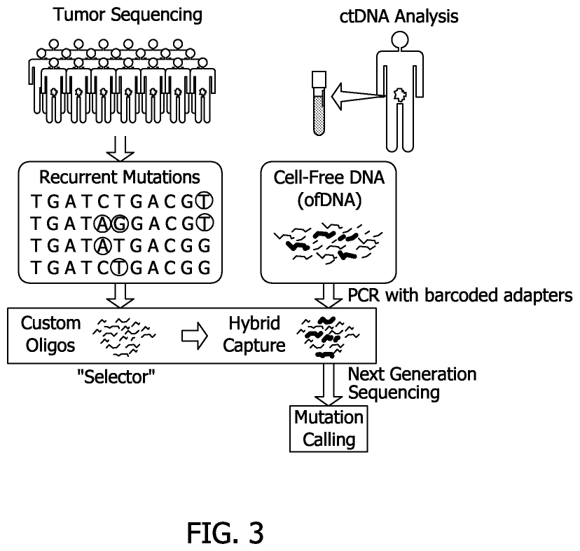

In various aspects, the genomic alterations associated with mCRPC may be monitored noninvasively in cell-free DNA using platforms such as targeted hybrid-capture next-generation sequencing (NGS). Use of such a platform may begin with a design of a hybrid-capture panel for cfDNA hybrid-capture and NGS based on analysis of publicly available whole exome and/or whole genome sequencing data to identify recurrent mutations in the cancer of interest, as illustrated in FIG. 3. Without being limited to any particular theory, the detection limit of the targeted hybrid-capture NGS platform may be affected by the absolute number of available cell-free DNA molecules in a given volume of peripheral blood, as well as PCR and sequencing errors (i.e. “technical” background). In some aspects, sensitivity and specificity may be significantly improved through the addition of duplex molecular barcodes to reduce PCR errors, and bioinformatics background error correction to reduce stereotypic technical noise.

In various aspects, the sensitivity of the targeted hybrid-capture NGS assay of the disclosure may be further enhanced to improve its analytical limit-of-detection by adjusting at least one or more parameters defining the protocol used to perform it. Non-limiting examples of suitable parameter adjustments include: (1) measuring larger volumes of plasma to increase the number of cfDNA molecules available for ligation; (2) further improving ligation conditions by optimizing volume, incubation time, temperature, enzyme type/source; (3) increasing sequencing depth per sample (i.e. increase from ˜2,000× to ˜4000× coverage in the sequencing space). In various aspects, the sensitivity of the targeted hybrid-capture NGS assay of the disclosure may be greater than about 20%. In various aspects, the sensitivity of the targeted hybrid-capture NGS assay may be greater than about 30%, greater than about 40%, greater than about 50%, greater than about 60%, greater than about 70%, greater than about 80%, or greater than about 90%.

Numerous published studies demonstrate the effectiveness of hybrid-capture NGS assays at identifying important prognostic biomarkers. By way of non-limiting example, targeted hybrid-capture NGS assays have been applied to localized lung cancer to monitor disease burden and predict disease-free survival after curative-intent treatment, and to identify patients at high risk for recurrence after radiotherapy. By way of other non-limiting examples, such assays have been applied to: metastatic lung cancer to identify treatment failure and resistance mechanisms to a third-generation tyrosine kinase inhibitor; to metastatic colorectal cancer to predict treatment failure and identify RAS pathway based resistance mechanisms to the first-generation tyrosine kinase inhibitor erlotinib; to leiomyosarcoma to identify mutations pre-treatment and track these mutations posttreatment; to diffuse large B cell lymphoma to monitor tumor evolution and predict chemotherapy response early; and to detect molecular residual disease (MRD) after definitive-intent bladder cancer treatment and localized lung cancer treatment.

In various aspects of the disclosure, cell-free DNA analysis may be used to detect resistance to AR-directed therapy and to identify high-risk metastatic prostate cancer patients. As illustrated in the examples below, a hybrid-capture NGS assay, which we refer to as EnhanceAR-Seq, significantly outperformed the standard CTC AR-V7 detection approach that is currently used clinically.

As described in the examples below, patients with detectable structural variations in AR or its enhancer developed primary resistance to AR-directed therapy over a relatively short follow-up period. AR/enhancer SV detection using the disclosed EnhanceAR-Seq assay was associated with significantly worse PFS, OS and decreased PSA response to treatment. In contrast, the Genomic Health CTC AR-V7 assay did not show any correlation with clinical outcomes. These results suggest that cell-free DNA detection of AR/enhancer structural variations is a more sensitive and accurate way to identify resistance to AR-directed therapy than the CTC AR-V7 assay used in clinical practice. A comprehensive analysis approach that measures both amplifications and genomic rearrangements in both the AR gene body and upstream enhancer was shown to identify high-risk disease much more sensitively than examining AR gene body amplifications alone.

Resistant patients identified by detection of AR/AR enhancer structural variations using the EnhanceAR-Seq assay as disclosed herein may be completely distinct from those with AR-V7 messenger RNA splice variations. Given these different mechanisms of resistance, one at the DNA level (which may be identified using the disclosed EnhanceAR-Seq assay), and the other at the mRNA/protein level (assessed using the Genomic Health CTC AR-V7 test), it may be valuable in cases to run both assays to more comprehensively assess multiple mechanisms of resistance.

In various additional aspects, the disclosed EnhanceAR-Seq assay may be used to detect genomic alterations in addition to the AR/AR enhancer alterations described herein. Additional genomic alterations detectable by the disclosed EnhanceAR-Seq assay include, but are not limited to, a TMPRSS2-ERG fusion, which was demonstrated to be detectable in 17% of a patient cohort in the examples below. These additional genomic alterations may be used as prognostic biomarkers and/or predictive biomarkers to diagnose and/or stratify various prostate cancer patients.

As illustrated in the examples below, the cell-free DNA analysis enabled by the disclosed EnhanceAR-Seq assay is useful for detecting structural variations in AR and its enhancer that reliably identify patients with primary resistance to AR-directed therapy. In addition, the EnhanceAR-Seq assay may be used to stratify patients based on progression-free survival and overall survival despite a short median follow-up time. A cell-free DNA analysis enabled by EnhanceAR-Seq may significantly improve the management of metastatic prostate cancer by opening the door to more personalized treatment approaches.

### EXAMPLES

The following examples are provided to illustrate various aspects of the disclosure.

### Example 1: Landscape of Somatic and Structural Alterations in mCRPC Patients

Somatic and structural alterations associated with metastatic castration-resistant prostate cancer patients have been conducted previously (see Quigley et al., Genomic Hallmarks and Structural Variation in Metastatic Prostate Cancer, Cell; 174(3); 758-769 (2018)).

Metastatic tumor biopsies were obtained from 101 mCRPC patients that had developed resistance following front-line treatment with androgen deprivation therapy (ADT). The biopsies were subjected to deep whole-genome and transcriptome analysis to investigate the genomic drivers of mCRPC comprehensively.

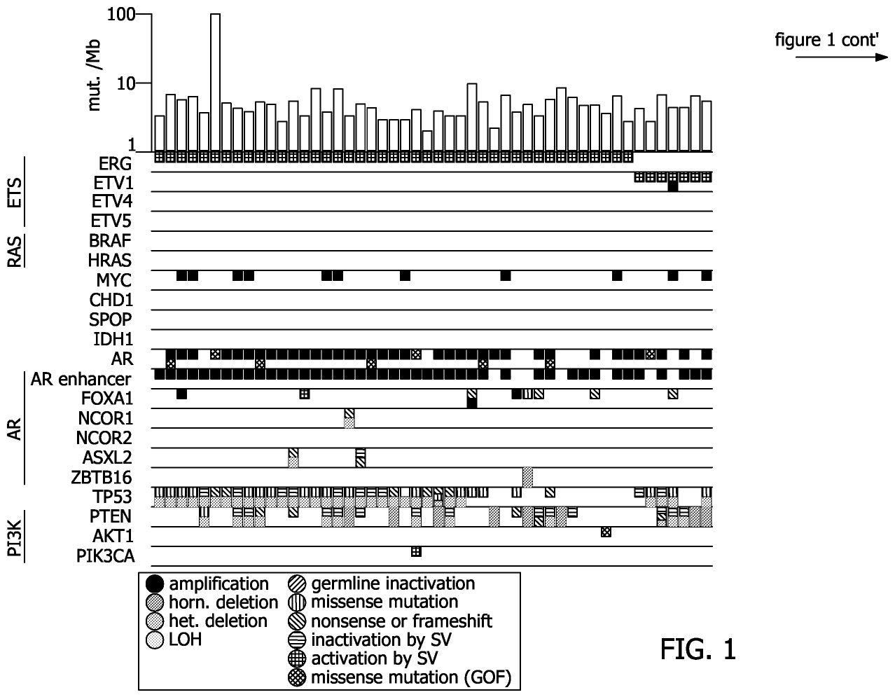

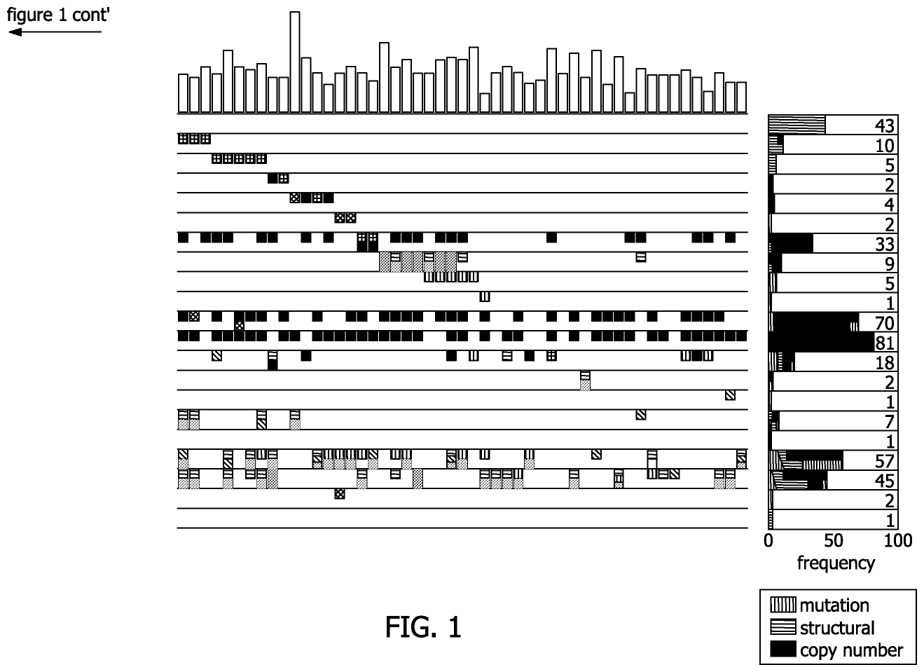

FIG. 1 summarizes a subset of the single nucleotide variants (SNVs) and SVs for 84 key mCRPC genes identified from the analysis of the biopsy samples described above. Somatic mutation frequencies were consistent with mCRPC genes previously identified using exome sequencing. Referring to FIG. 1, the most frequent events included amplifications involving AR and MYC, inactivating SVs targeting and resulting in decreased expression of tumor suppressor genes including TP53, PTEN, RB1, and CHD1, and activating gene fusions involving the ETS oncogenic transcription factor family and the druggable targets AXL and BRAF.

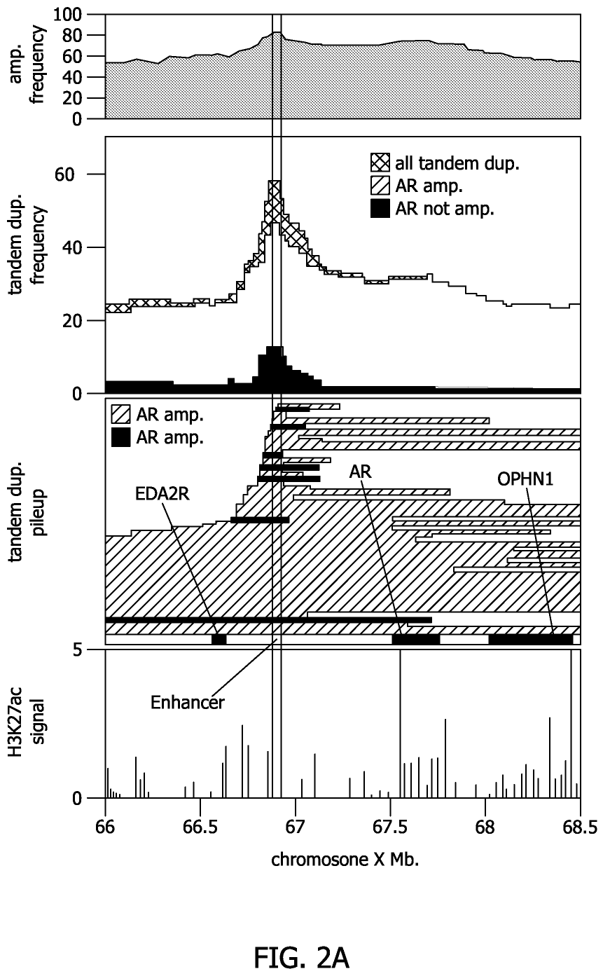

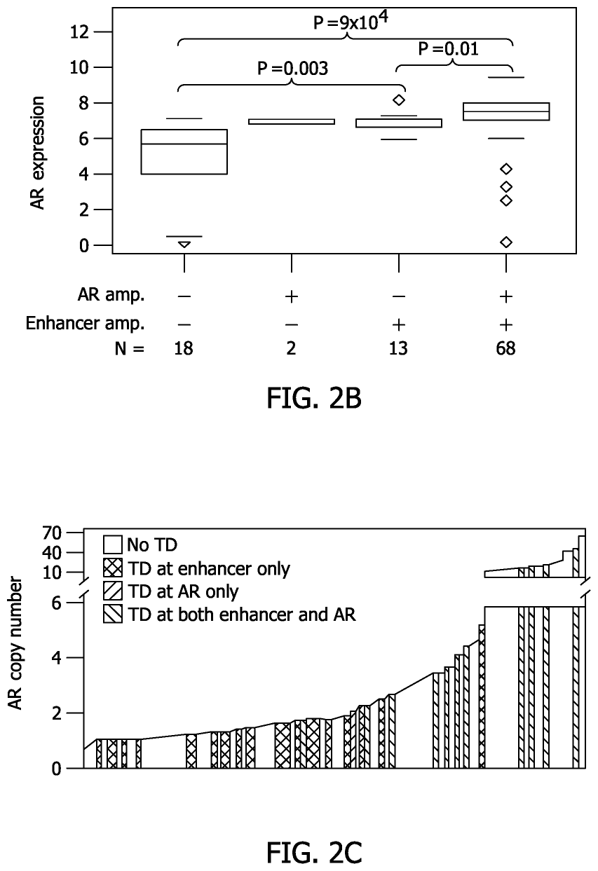

To explore the etiology of SVs in prostate cancer, alterations associated with different SV categories were identified (see FIG. 8). Classes of SVs that were linked to distinct DNA repair deficiencies were observed. This included associations of CDK12 mutations with tandem duplications, TP53 inactivation with inverted rearrangements and chromothripsis, and BRCA2 inactivation with deletions (see FIGS. 2A-2C). Overall, these findings confirmed that DNA repair defects in mismatch repair and homologous recombination may produce genomic scars in metastatic prostate cancer. The frequency of mutations in genes responsible for DNA damage repair was also assessed. Inactivating germline alterations were present in the DNA repair genes BRCA2 and ATM in 4% of samples. Somatic alterations alone accounted for five of the eight cases of biallelic BRCA2 inactivation, with all five tumors carrying biallelic CDK12 inactivation. Biallelic BRCA2, CDK12 and ATM inactivating mutations were mutually exclusive, and the total frequency of biallelic BRCA2, CDK12, and ATM inactivation was 15%. Overall, these results highlight the rationale for using poly (ADP-ribose) polymerase (PARP) inhibitors to treat mCRPC patients with DNA damage repair defects.

One of the most prevalent events was amplification of AR in 70% of patients, which was significantly associated with elevated AR mRNA expression (p=9×10-8). This result was consistent with earlier findings that AR amplification was rare in primary PCa37 but common in mCRPC17 and was a major mechanism of resistance to androgen deprivation therapy (ADT). However, the integrated analysis of the whole genome and transcriptome across a large population of mCRPC patients revealed the amplification of a distal enhancer region 624 kb upstream of AR. This AR enhancer tandem duplication was present in 81% of men, and 85% of men had either an amplification or a pathogenic activating AR mutation, as illustrated in FIGS. 2A, 2B, and 2C.

The results of these experiments support the model that amplification at the putative enhancer locus resulted in increased AR expression. In 13% of men, this putative enhancer amplification was present without alterations in the AR gene itself, as illustrated in FIG. 2B. This finding suggested that DNA copy number gain affecting this locus, commonly by tandem duplication, may be a frequent mechanism by which prostate tumor cells initially developed ADT resistance.

The AR enhancer amplification was not observed in localized non-resistant PCa, suggesting it was acquired during cancer progression while on ADT, similar to AR gene amplification. Furthermore, intragenic SVs (deletions, inversions and translocations) were identified that potentially truncate the AR ligand binding domain in 7% of mCRPC patients. Taken together, the results of these experiments suggested that AR SVs, including amplification of the distal enhancer, may serve as biomarkers for castration resistance.

### Example 2: mCRPC EnhanceAR-Seq Assay

To develop EnhanceAR-Seq, an ultra-sensitive targeted hybrid-capture NGS assay for mCRPC patients with superior ability to detect structural variations, copy number alterations, fusions/rearrangements, single nucleotide variants and insertions/deletions, and identify therapeutic resistance early, the following experiments were conducted.

To develop the EnhanceAR-Seq ctDNA assay for mCRPC patients, a Selector was designed that included 84 genes (˜400 kb total) harboring SNV or SV events; 48 of the selected genes had been previously observed to be affected by copy number alteration. The Selector panel targeted the AR enhancer region and its flanking sequence, AR full-length gene body, TMPRSS2 and ERG exons and introns involved in TMPRSS2-ERG fusion breakpoints, and the exons of genes frequently mutated in mCRPC based on the results of the whole genome study described in Ex. 1 above. The 84 genes of the Selector further ensured that SVs involving AR and the AR enhancer were monitored, as well as lower probability genomic events from the results of the whole genome study described in Ex. 1 above. The NimbleGen SeqCap EZ platform (Roche) was used for targeted hybrid capture, which was applied to plasma with matched germline samples from 20 high-risk prostate cancer patients, 12 with mCRPC and 8 with metastatic hormone sensitive PCa (mHSPC), as summarized in Table 1 below.

TABLE 1
Patient and tumor characteristics and noninvasive biomarker status.

| | | | | Clinically | AR enhancer | AR-V7 | |
|---|---|---|---|---|---|---|---|
| Patient | Age | | | [symbol] | amplified in | [symbol] | |
| ID | (y) | Status | Line of Rx | resistant? | [symbol] | assay | Race/Ethnicity |
| PB-032 | 54 | mHSPC | Initial ADT | No | NEG | | Caucasian |
| PB-069 | 75 | mHSPC | Initial ADT | No | NEG | | Caucasian |
| PB-077 | 70 | mCRPC | 2 | Yes | POS | | Caucasian |
| PB-078 | 59 | mCRPC | 5 | No | NEG | NEG | Caucasian |
| PB-087 | 62 | mCRPC | 3 | Yes | POS | NEG | Caucasian |
| PB-088 | 78 | mCRPC | 3 | Yes | POS | NEG | Caucasian |
| PB-106 | 62 | mCRPC | 4 | Yes | POS | NEG | Caucasian |
| PB-132 | 69 | mCRPC | 2 | Yes | POS | | Caucasian |
| PB-140 | 76 | mCRPC | 4 | Yes | POS | | Caucasian |
| PB-169 | 94 | mHSPC | Initial ADT | No | NEG | POS | Caucasian |
| PB-174 | 70 | mCRPC | 4 | Yes | POS | NEG | African American |
| PB-188 | 67 | mHSPC | Initial ADT | Yes | POS | | Caucasian |
| PB-202 | 74 | mCRPC | 2 | No | NEG | NEG | Caucasian |
| PB-203 | 57 | mCRPC | 2 | Yes | POS | NEG | Caucasian |
| PB-204 | 80 | mCRPC | 2 | Yes | POS | | Other/Non- |
| | | | | | | | [symbol] |
| PB-208 | 72 | mHSPC | Initial ADT | No | POS | | Caucasian |
| PB-210 | 50 | mCRPC | 2 | No | NEG | | African American |
| PB-226 | 88 | mCRPC | 3 | Yes | NEG | NEG | Caucasian |
| PB-239 | 55 | mHSPC | Initial ADT | No | NEG | NEG | Caucasian |
| PB-242 | 74 | mHSPC | Initial ADT | No | NEG | | Caucasian |

[symbol]

indicates data missing or illegible when filed

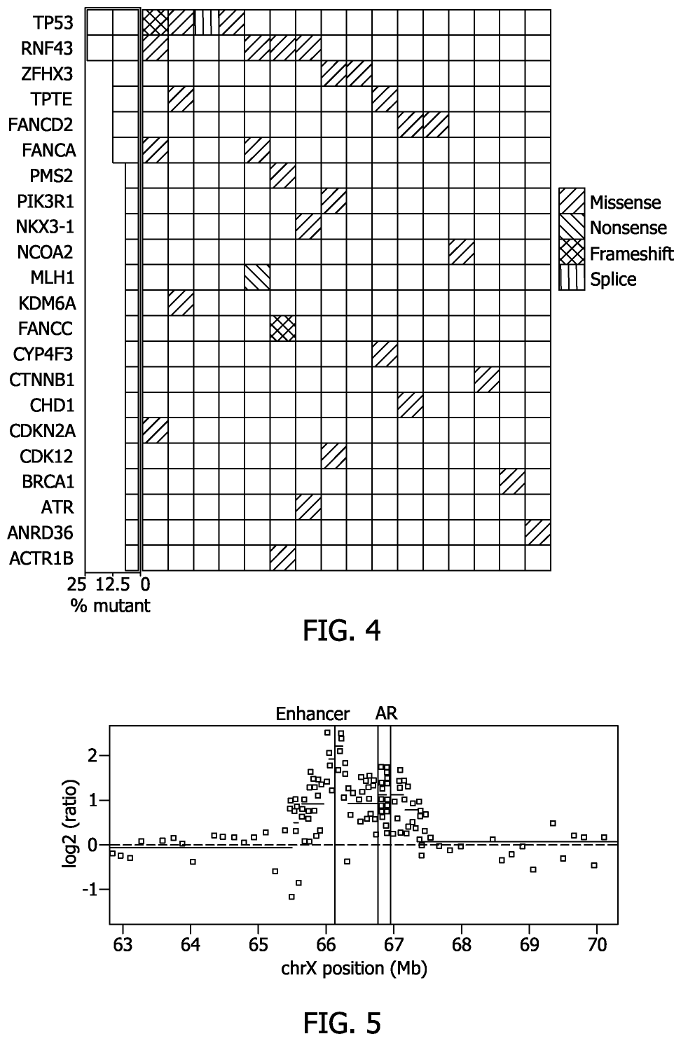

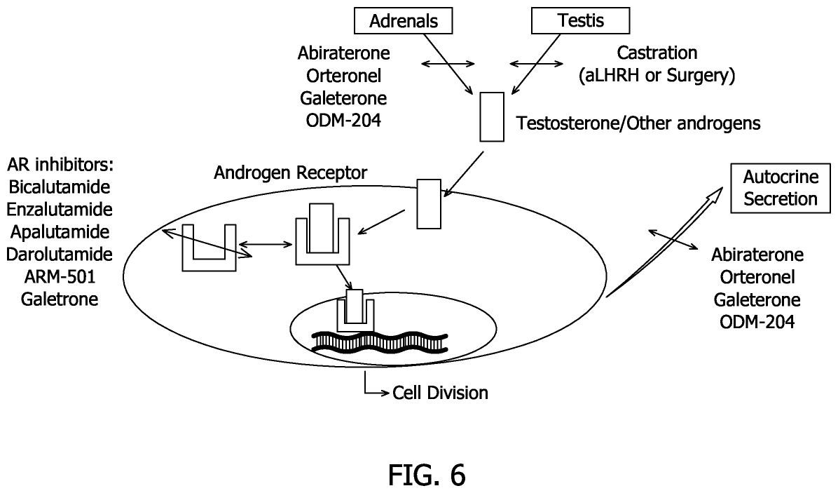

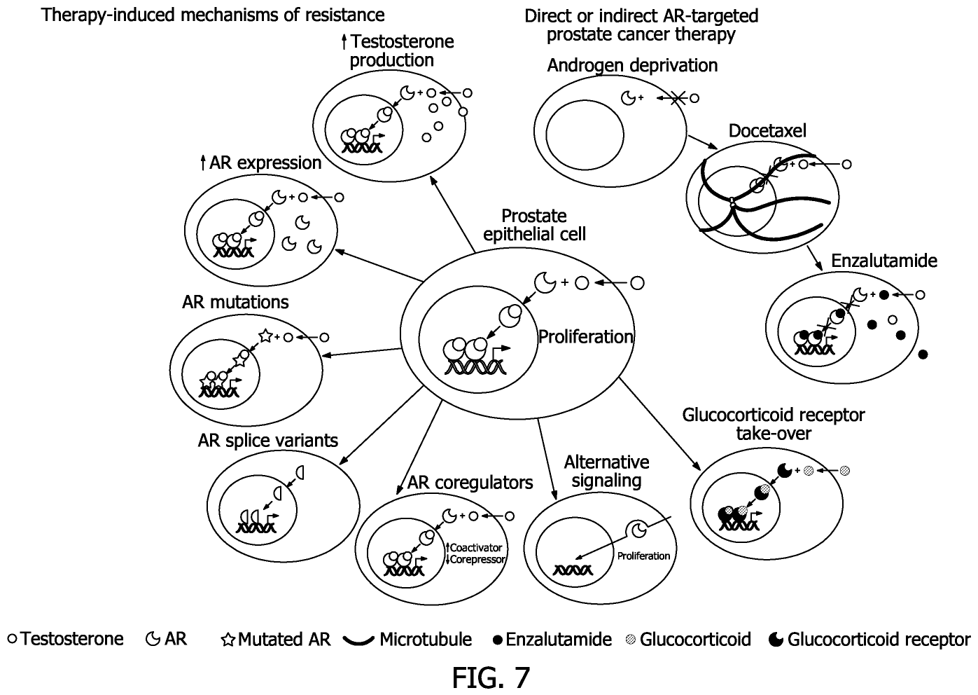

Library preparation was performed using duplex barcoded adapters. NGS was completed using an Illumina HiSeq4000 with 2×150 bp paired-end reads, dedicating ˜70 million reads per sample. A median coverage in the Selector space of ˜2,000× per sample was achieved. In addition, a median of 1.5 nonsynonymous SNVs and indels in plasma were identified that were not observed in germline, that most commonly affected TP53 and RNF43, as summarized in FIG. 4.

As illustrated in one exemplary patient in FIG. 5, the AR enhancer was amplified in 11 of the patients (55%) and AR enhanced amplification was absent in the remaining 9 patients. 10 of 11 patients detected with AR enhancer amplification had prostate cancer refractory to AR-directed therapy, including a patient previously classified as hormone-sensitive with newly diagnosed castration resistance while on first-line ADT. 8 of the 9 patients detected with no AR enhancer amplification apparent were classified as responsive to ADT or AR-directed therapy. Positive and negative predictive values for identifying refractory castration resistance were 91% and 89%, respectively, using the ctDNA assay. By comparison, the corresponding positive and negative predictive values for the existing standard-of-care ARV7 CTC assessment was 0% and 33%, respectively.

The results of these experiments suggest that tracking amplification of the AR enhancer in cell-free DNA can identify castration resistance refractory to AR-directed therapy with enhanced sensitivity and specificity relative to the clinically validated AR-V7 assay. This enhanced sensitivity and specificity may enable identification of castration resistance refractory to AR-directed therapy more robustly and earlier than the AR-V7 assay.

### Example 3: Validation of mCRPC EnhanceAR-Seq Assay in African American Population

To validate the EnhanceAR-Seq ctDNA assay disclosed herein for a cohort of African-American mCRPC patients, the following experiments will be conducted.

The incidence of distal AR enhancer amplification and other AR structural variations (SVs) in a cohort of African-American (AA) prostate cancer patients was investigated. AA patients are 1.7× more likely to be diagnosed with, and 2.3× more likely to die from prostate cancer than Caucasian (CA) patients. The understanding of the SVs underlying mCRPC in AA men, based on the results of Ex. 2 demonstrating stereotypic distal enhancer amplification in mCRPC remains incomplete, because the patient population of Ex. 2 included <5% AA patients. The cohort used in previously reported results reports related to the standard-of-care ARV7 CTC assessment included less than 6 AA patients. This disparity will be addressed by performing targeted deep sequencing in a cohort of AA men in order to characterize genomically AR SVs, including amplification of the upstream distal enhancer region as described in Ex. 1 and Ex. 2.

Ten samples were collected from AA mCRPC patients and additional samples will be prospectively collected by various genitourinary (GU) medical oncologists. Metastatic tumor tissue from standard-of-care biopsies will be collected, along with contemporaneous plasma and plasma-depleted whole blood. Archival metastatic biopsy tissue will also be considered for collection, provided patients have continued follow-up and can provide peripheral blood to expedite the collection of appropriate samples. Biopsies will be collected as fresh frozen or placed in formalin and processed using standard clinical pathology procedures. Bone biopsies will undergo standard EDTA decalcification. Samples will then be processed and areas of >50% tumor cellularity will be identified for nucleic acid isolation. All tissue, plasma and plasma-depleted whole blood will be processed for DNA extraction using previously published methods known in the art. Clinical data will be collected and stored in a secure database.

The samples acquired as described above will be subjected to targeted tumor sequencing and analysis using the mCRPC Selector described in Ex. 2 and analysis using the analysis protocol as described in Ex. 2. Simultaneously, EnhanceAR-Seq with the mCRPC Selector will be used to identify genomic alterations in contemporaneous matched plasma samples (using plasma-depleted whole blood as a germline reference) as described in Ex. 2. The incidence of AR SVs within this AA cohort will be evaluated, including amplification of the upstream AR enhancer found as described in Ex. 1 and Ex. 2 above. The full genomic breadth of the Selector space described in Ex. 2 will be used to identify other genomic aberrations that will be assessed in both tumor and matching plasma. The results from this AA cohort will be compared to the previous results from Ex. 2, obtained from a predominantly CA cohort.

The previous cohort of predominantly CA mCRPC patients described in Ex. 2 included AR enhancer amplifications or pathogenic activating AR mutations in 85% of all patients. Assuming a similar prevalence of these genomic alterations in the current experiment, based on a one-sample non-inferiority test with 0.1 margin, 101 tumor biopsies with matching plasma samples will be obtained and analyzed. This calculation was based on a one-sample one-sided exact test with power of 80% and significance level of 0.05. A one-sample test using Wilcoxon signed-rank test will be used to compare the results of the current experiment with the AA cohort proposed cohort to results of the experiment of Ex. 2. Correlations of continuous variables between tumor biopsies and plasma samples will be assessed by Spearman rank correlation. McNemar's test will be conducted to statistically test the binary paired results among tumor and plasma samples. Chi-square or Fisher's exact test will be used for contingency table tests.

The results of this experiment will identify AR SVs characteristic of high-risk mCRPC in African American patients as previously obtained for the cohort of Ex. 2. Stereotypic amplification of the distal AR enhancer in a high percentage of these patients will be observed. The breadth of the mCRPC Selector will be utilized to perform exploratory comparisons beyond AR biomarkers to identify the potential for use of novel targeted therapies (i.e., AXL inhibitor, PARP inhibitor) in these patients.

### Example 4: Protocol of a Comparison of EnhanceAR-Seq Cell-Free DNA Sequencing to AR-V7 Assay

To compare the efficacy of the EnhanceAR-Seq cell-free DNA assay at identifying lack of response to AR-directed treatment in mCRPC patients to standard-of-care assays that detect AR-V7 mutation in patient CTCs, the following experiments were conducted.

While detection of the AR-V7 mutation in patient CTCs correlates with worse prognosis and a lack of response to AR-directed treatment, it is only detected in ˜10-20% of mCRPC patients following first-line treatment. In contrast, stereotypic amplification of a distal enhancer region 624 kb upstream from AR was observed in 81% of mCRPC cases as described in Ex. 2.

The EnhanceAR-Seq based cell-free DNA assay for tracking AR-V7 mutations and other AR SVs as descried in Ex. 2 was optimized and evaluated as a more sensitive predictive biomarker for AR-refractory mCRPC than the clinically validated AR-V7 assay. By sensitively detecting AR-refractory mCRPC, clinicians will be able to more reliably identify these patients and nimbly pivot to non-AR focused treatments (such as immunotherapy or chemotherapy) when AR-directed drugs like abiraterone and enzalutamide lose efficacy.

The current AR-V7 clinical test is used in progressive mCRPC patients who have previously received treatment with at least one AR-directed therapy (e.g. enzalutamide or abiraterone). After one line of AR-directed therapy, sensitivity of the CTC-based assay is ˜18%, which is quite low compared to previously published findings of AR alterations in mCRPC and the results described in Ex. 2. Plasma from mCRPC patients were collected and, in parallel, the clinically validated CTC based AR-V7 assay (run through Genomic Health/Epic Sciences) in its approved space (after one or more lines of AR-directed therapy) was utilized. Patients were identified and selected by oncologists specializing in GU malignancies and treatment of mCRPC patients. AR-V7 testing was sent from the clinic as a standard-of-care clinical test. De-identified whole blood collected in EDTA tubes was processed for plasma (for cfDNA) and plasma-depleted whole blood (for germline). After centrifugation at 1800×g for 10 minutes, plasma was removed using filter-tips, then spun a second time at 1800×g for 10 minutes (to reduce the risk of genomic DNA contamination of cfDNA), frozen at −80° C., then stored in liquid nitrogen. A 1 mL aliquot of the remaining plasma-depleted whole blood (from the initial spin) was also frozen and stored for germline DNA extraction. Clinical information was collected and stored in a secured database.

Analysis was performed as described in Ex. 2 by applying the mCRPC Selector described in Ex. 2 on plasma (with matched germline derived from plasma-depleted whole blood) for the patients identified as described above. CTC ARV7 status was concurrently assessed at matched timepoints using the clinically validated test from Genomic Health/Epic Sciences, per clinical standard-of-care. The results were compared with the corresponding results from the AR-V7 assay, and clinical outcomes were evaluated in the context of both results.

It is estimated that ˜58% of mCRPC patients become resistant to AR-directed therapy. EnhanceAR-Seq assay AR positivity rate in mCRPC (regardless of line of treatment) is estimated to be ˜80-90% based on the ctDNA data described in Ex. 2, as well as previously published tumor sequencing data. Therefore, a kappa coefficient of 0.2-0.40 was regarded as fair, 0.4˜0.6 as moderate, and 0.6˜0.8 as substantial agreement. The sample size of 50 patients allowed 80% power to test a hypothesized kappa of at least 0.6 against a fair kappa with 82% power at a 5% level. For rPFS, a median rPFS of about 2.1 and 14.5 months would be expected for assay positive and negative mCRPC, respectively. Furthermore, the population size of 50 patients allowed for 80% power to detect such a survival difference with a 12-month accrual period and 24-month study duration.

The results from patient tumors were used as the benchmark standard. Concordance between assay results from plasma against assay results from tumors (collected on the same patients) were gauged by Cohen's kappa with 95% bootstrapping-based confidence intervals (CIs). Diagnostic test properties such as sensitivity and specificity were calculated. McNemar's test was conducted to statistically test the paired results among tumor and plasma samples. Assay results were associated with clinical outcomes including treatment response, radiographic progression free survival (rPFS) and overall survival. The Kaplan-Meier method was used to estimate empirical survival probabilities by assay results with survival differences compared by log-rank test. Cox proportional hazards models were used to estimate unadjusted hazard ratios (HRs) and adjusted HRs from multivariate Cox models with 95% CI when clinical-pathological variables were included. Fisher's exact test was used to test the association of the results with treatment response while unadjusted and adjusted odds ratios were estimated from univariate and multivariate logistic regression models. The clinically used Genomic Health/Epic Sciences AR-V7 assay was similarly analyzed. The performance between the mCRPC EnhanceAR-Seq assay described in Ex. 2 as applied to tumor and plasma, and the Genomic Health/Epic Sciences AR-V7 assay, was compared using area under the ROC curve for logistic regression model and Harrell's C-index for Cox model.

The results of these experiments demonstrated that the AR structural variations detected using the mCRPC EnhanceAR-Seq assay described in Ex. 2 (namely amplification of the distal AR enhancer) serve as superior biomarkers for a larger proportion of mCRPC patients compared to the CTC AR-V7 assay. The mCRPC Selector described in Ex. 2 may be utilized to perform exploratory comparisons beyond AR biomarkers to identify the potential for novel targeted therapies in some patients (i.e., AXL inhibitor, PARP inhibitor).

### Example 5: Efficacy of EnhanceAR-Seq Cell-Free DNA Sequencing in mCRPC Patients Prior to First-Line Treatment

To evaluate genomic aberrations including AR SVs in the cell-free DNA of mCRPC patients prior to first-line treatment, the following experiments will be conducted.

The AR-V7 assay's sensitivity is known to be extremely low prior to first-line treatment for castration resistance (˜3%), thus insurance coverage for this test is restricted in this setting and it is not clinically approved for use at this early timepoint. Given the poor prognosis associated with AR-refractory mCRPC (˜5.5 mo median survival), it is critical to identify these patients as soon as possible, prior to first-line treatment, in order to enable earlier clinical intervention before the disease has progressed substantially.

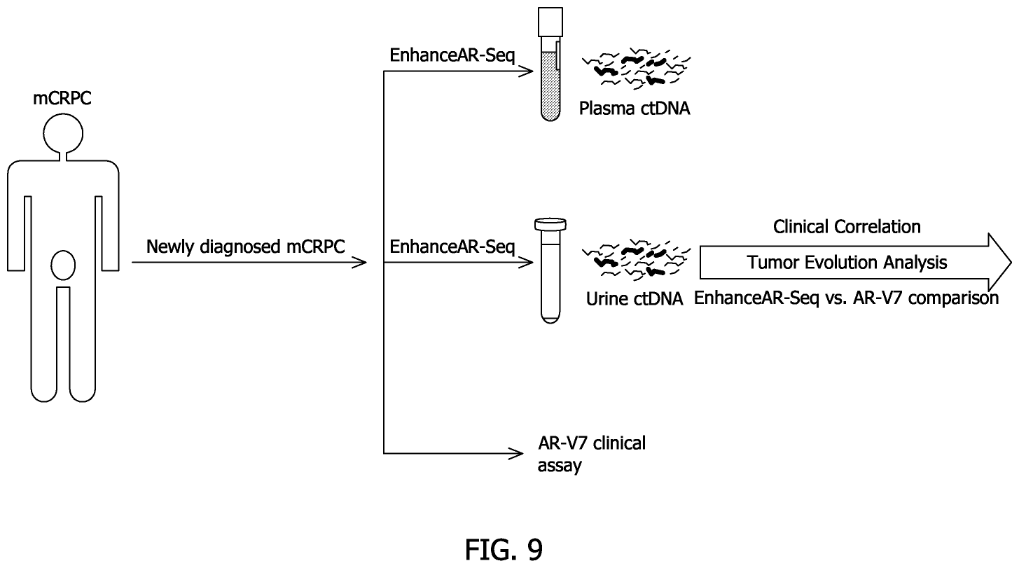

Critical preliminary data characterizing the timing and evolution of mCRPC genomic aberrations will be established by evaluating mCRPC patients prior to first-line treatment using the mCRPC EnhanceAR-Seq assay described in Ex. 2. A schematic diagram illustrating the experimental procedure is provided as FIG. 9. Blood plasma will be collected from newly diagnosed mCRPC patients prior to treatment and the mCRPC EnhanceAR-Seq assay described in Ex. 2 will be applied to establish the incidence of genomic alterations including distal AR amplification. These findings will be correlated with clinical outcomes, and will serve as preliminary data for powering a larger subsequent predictive biomarker study. This predictive biomarker study will enable identification of AR-refractory mCRPC much earlier than is currently possible, which will help improve survival for this dangerous form of prostate cancer.

mCRPC EnhanceAR-Seq will be applied to plasma cell-free DNA collected from newly diagnosed mCRPC patients. Prospective collection of plasma with matched germline will be performed as described above in Ex. 4 for 100 newly diagnosed mCRPC patients who have not yet been treated with an AR-directed therapy (i.e. mHSPC patients with PSA progression on ADT). EnhanceAR-Seq analysis will be performed on all samples using the mCRPC Selector as described in Ex. 2. The low prevalence of AR-V7 positivity in the treatment-naïve mCRPC population (˜3%) limits testing utility at this early timepoint.

The results of the mCRPC EnhanceAR-Seq analysis and the noninvasive AR SV detection assay will be correlated with clinical outcomes. In addition, the full Selector space described in Ex. 1 will be utilized in an expanded EnhanceAR-Seq analysis to characterize the genomic aberrations that occur at the inception of castration resistance and correlating these genomic aberrations with clinical outcomes. Finally, a historic rather than direct comparison will be made between the EnhanceAR-Seq results and published data using the CTC AR-V7 assay in the first-line setting as it will not be possible to measure AR-V7 as part of standard-of-care.

Since the assay positivity rate is assumed to be ˜50% lower among untreated mCRPC (although the preliminary data suggests the true positive rate may be much higher), a sample size of N=100 is justified with similar specifications as described in Ex. 4. This will enable more than 90% for kappa testing and 80% power for detecting a survival difference at a time-point, testing on a HR of 0.5˜0.6 when varying the median rPFS of assay-positive patients from 5 to 12 months. Assay results will be associated with clinical outcomes including treatment response, rPFS and overall survival as described in Ex. 4. Subset analyses will be performed (e.g., on responding or resistant patients). The random forest algorithm will be applied to examine the importance of variant allele fractions of the 84 genes in the EnhanceAR-Seq Selector to clinical outcomes, and to obtain prediction accuracy based on an ensemble of predictive trees. Last, the EnhanceAR-Seq results were compared to previously published data utilizing the CTC AR-V7 assay in the first-line setting by comparing Z-scores of test results, and performing ROC analyses and calculating AUC and Youden's J statistic for sensitivity and specificity.

The results of the EnhanceAR-Seq assay will detect relevant genomic alterations (i.e. distal AR enhancer amplification) at a high frequency in newly diagnosed AR-refractory mCRPC cases. The results will further enable clinical translation of the EnhanceAR-Seq assay to an earlier and more clinically meaningful disease state than the AR-V7 assay and potentially direct first-line non-ADT treatment.

### Example 6: Development of EnhanceAR-Seq Cell-Free DNA Sequencing for mCRPC Patients

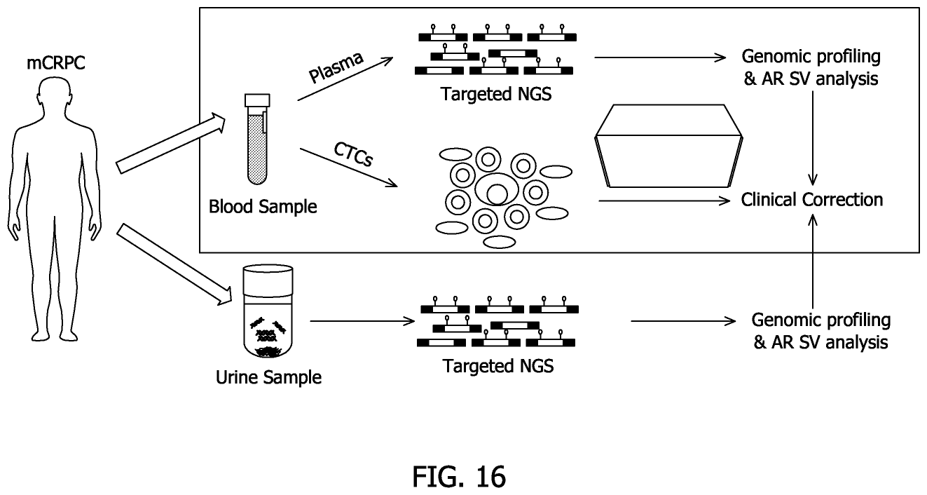

To develop a customized gene panel catered to metastatic prostate cancer along with a new bioinformatics pipeline, the following experiments were conducted. An overview of these experiments are illustrated schematically in FIG. 16.

41 patients (see Table 3 and Table 4) were prospectively enrolled with a median age of 65 years, and an ECOG performance status ranging between 0 and 1. In addition, 36 healthy normal donors were enrolled with epidemiologic characteristics available on 24 of these (both plasma and plasma-depleted whole blood collected) to assess assay specificity.

TABLE 3
Patient characteristics

| Patient | Age | | | Patient | Age | | |
|---|---|---|---|---|---|---|---|
| ID | (y) | Status | Race | ID | (y) | Status | Race |
| PB-032 | 54 | CRPC | Caucasian | PB-239 | 55 | mHSPC | Caucasian |
| PB-069 | 75 | mHSPC | Caucasian | PB-241 | 62 | mHSPC | Caucasian |
| PB-077 | 70 | mCRPC | Caucasian | PB-242 | 74 | mHSPC | Caucasian |
| PB-078 | 59 | mCRPC | Caucasian | PB-258 | 84 | mCRPC | Caucasian |
| PB-079 | 78 | mCRPC | Caucasian | PB-046 | 61 | mCRPC | Caucasian |
| PB-087 | 62 | mCRPC | Caucasian | PB-142 | 58 | mCRPC | Caucasian |
| PB-088 | 78 | mCRPC | Caucasian | PB-163 | 66 | mCRPC | African American |
| PB-106 | 62 | mCRPC | Caucasian | PB-183 | 66 | mCRPC | Caucasian |
| PB-108 | 64 | Small cell | Caucasian | PB-270 | 64 | mCRPC | African American |
| PB-132 | 69 | mCRPC | Caucasian | PB-282 | 76 | mCRPC | African American |
| PB-140 | 76 | mCRPC | Caucasian | PB-281 | 61 | mHSPC | Caucasian |
| PB-169 | 94 | mCRPC | Caucasian | PB-196 | 64 | mCRPC | Caucasian |
| PB-174 | 70 | mCRPC | African American | PB-304 | 89 | mCRPC | African American |
| PB-177 | 73 | mHSPC | Caucasian | PB-306 | 61 | mHSPC | African American |
| PB-188 | 67 | mCRPC | Caucasian | PB-307 | 65 | mHSPC | Caucasian |
| PB-202 | 74 | mCRPC | Caucasian | PB-251 | 88 | mHSPC | Caucasian |
| PB-203 | 57 | mCRPC | Caucasian | PB-314 | 72 | mCRPC | Caucasian |
| PB-204 | 80 | mCRPC | Other/Non-Hispanic | PB-206 | 72 | mCRPC | Caucasian |
| PB-208 | 72 | mHSPC | Caucasian | PB-319 | 68 | mCRPC | Caucasian |
| PB-210 | 51 | mHSPC | African American | PB-322 | 78 | mHSPC | Caucasian |

| | | | |
|---|---|---|---|
| PB-226 | 88 | mCRPC | Caucasian |

TABLE 4
Summary of patient characteristics

| Variable | Number | Percentage |
|---|---|---|
| Age (yrs) | 69.8 | |
| Median (Range) | 51-94 | |
| Sernm PSA Level (ng/dl) | 145 | |
| Median (Range) | 0.1-1343 | |
| Gleason Score | | |
| 6 | 2 | 5% |
| 3 + 4 | 2 | 5% |
| 4 + 3 | 5 | 12% |
| 4 + 4 | 6 | 15% |
| ≥9 | 19 | 46% |
| Not Known | 6 | 15% |
| ECOG Performance | | |
| 0 or 1 | 32 | 78% |
| 2 | 9 | 22% |
| AR-V7 Test (N = 22) | | |
| Negative | 21 | 95% |
| Positive | 1 | 5% |

A liquid biopsy cell-free DNA technique (EnhanceAR-Seq) was developed to identify resistance to AR-directed therapy more sensitively than is possible with the current standard-of-care CTC AR-V7 assay. EnhanceAR-Seq was configured to make use of a customized gene panel catered to metastatic prostate cancer along with a bioinformatics pipeline.

The NimbleGen SeqCap EZ platform (Roche) was used for targeted hybrid capture within plasma samples with matched plasma-depleted whole blood germline samples from the 41 high-risk prostate cancer patients, and 24 healthy donor patients with matched plasma and plasma-depleted whole blood samples. An additional 12 healthy donor plasma samples were analyzed for bioinformatic background error correction. Library preparation was performed using a workflow with duplex barcoded adapters. NGS was then performed on an Illumina HiSeq4000 with 2×150 bp paired-end reads, with 12 samples sequenced per lane, dedicating ˜60 million reads per sample.

The hybrid capture gene panel included 84 relevant genes (˜400 kb total), summarized in Table 1, that have been shown to harbor genomic alterations in metastatic castration-resistant prostate cancer, as described in Ex. 1. A map of these genomic alterations are shown illustrated in FIG. 8. The gene panel was designed to target the AR enhancer region and its flanking sequence, AR full-length gene body (see FIG. 11 and FIG. 12), TMPRSS2 and ERG exons, introns involved in TMPRSS2-ERG fusion breakpoints (see FIG. 13 and FIG. 15), and the exons of genes frequently mutated in mCRPC based on the whole genome analysis as described in Ex. 1. The EnhanceAR-Seq assay quantified SVs in the upstream AR enhancer in patient samples, given the stereotypic AR enhancer amplifications identified in a high proportion of metastatic castration resistant prostate cancer patients as described in Ex. 2. In addition, copy number alterations and rearrangements (collectively referred to as “structural variations”) were tracked within AR and its enhancer.

The EnhanceAR-Seq assay was applied to blood plasma with matched plasma-depleted whole blood germline samples from the 41 prospectively enrolled patients with metastatic prostate cancer who were treated with AR-directed therapy, as well as the 24 heathy donors with plasma and plasma-depleted whole blood samples. Copy number alterations, genomic rearrangements, single nucleotide variants, and insertions/deletions in a set of 84 genes relevant to metastatic prostate cancer were quantified. The EnhanceAR-Seq assay was also used to analyze plasma samples obtained from an additional 12 healthy donors to reduce bioinformatic background error.

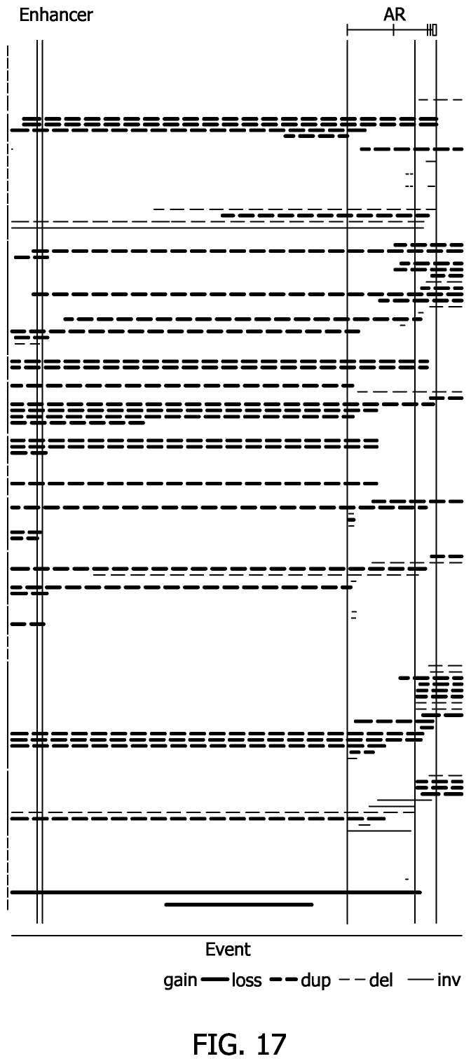

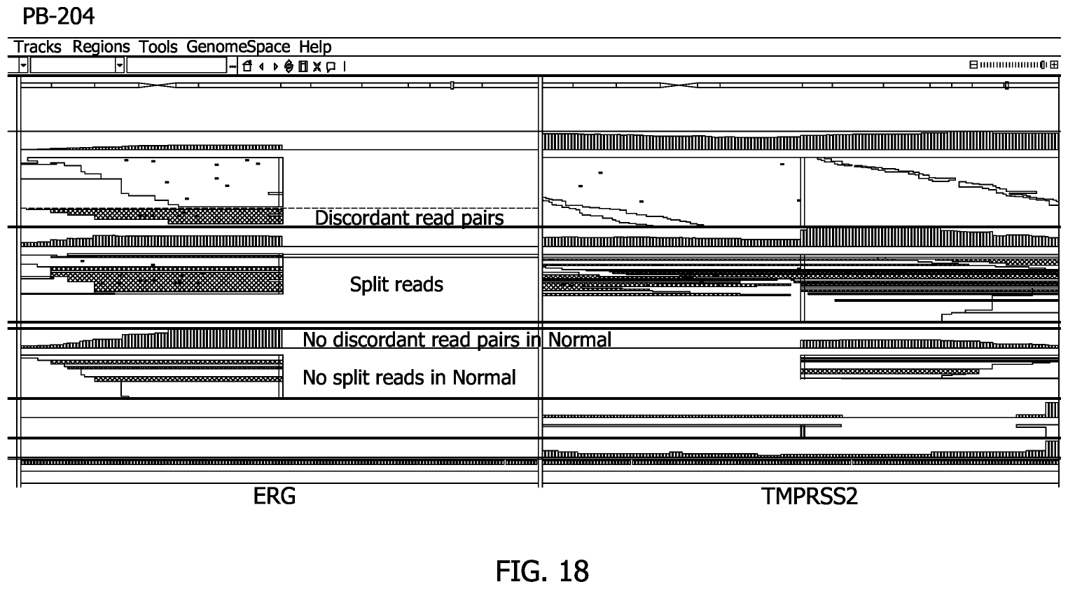

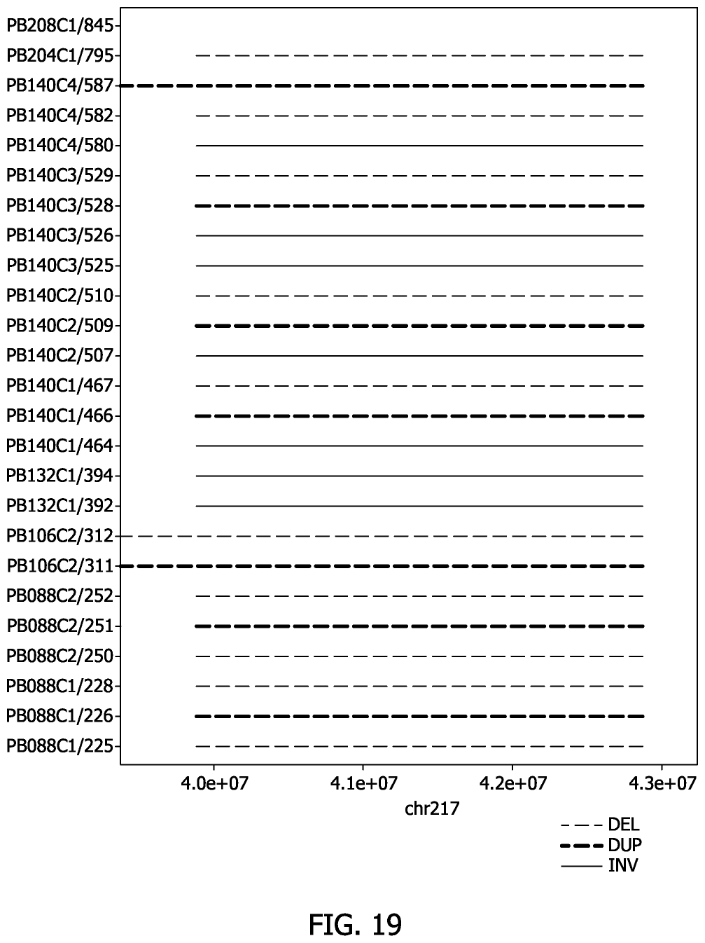

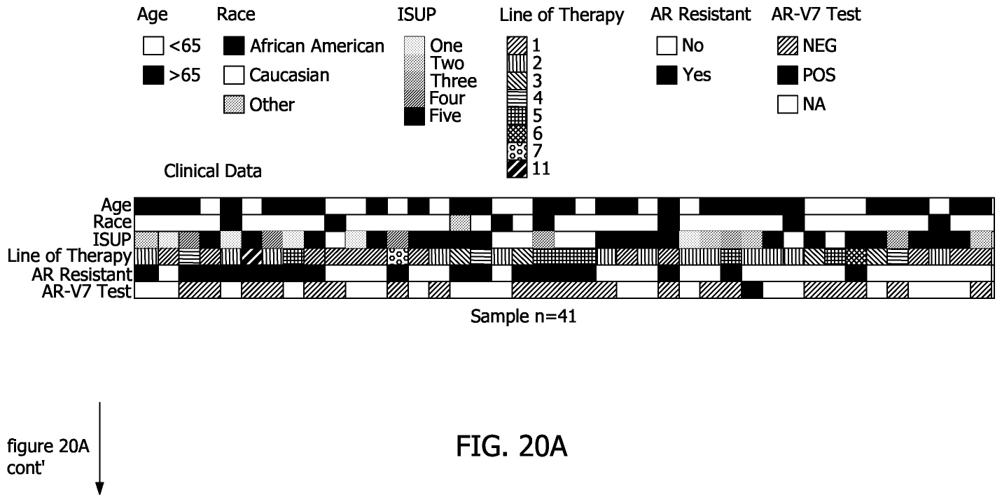

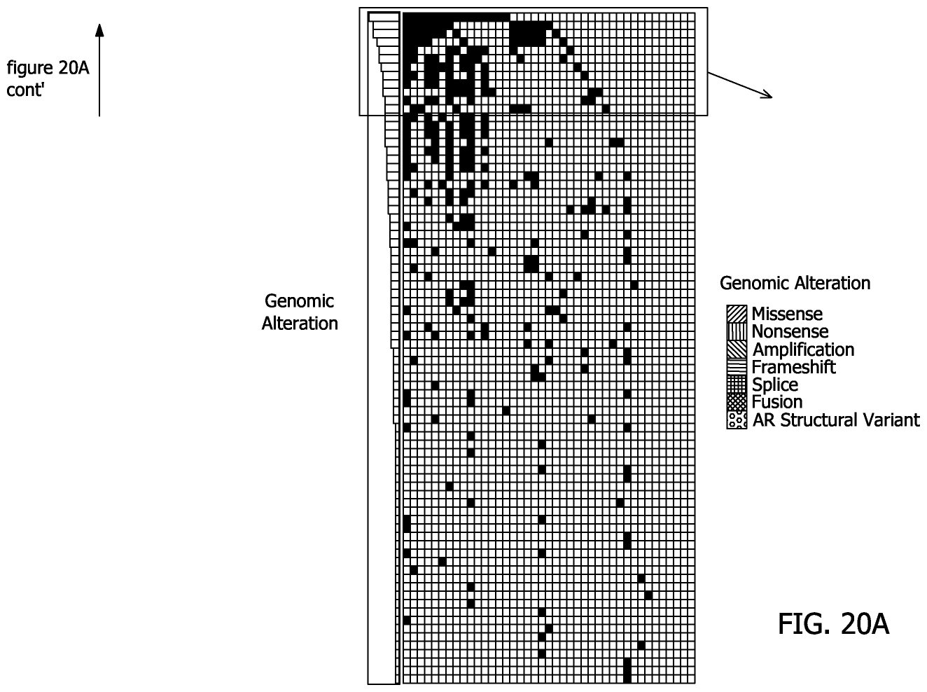

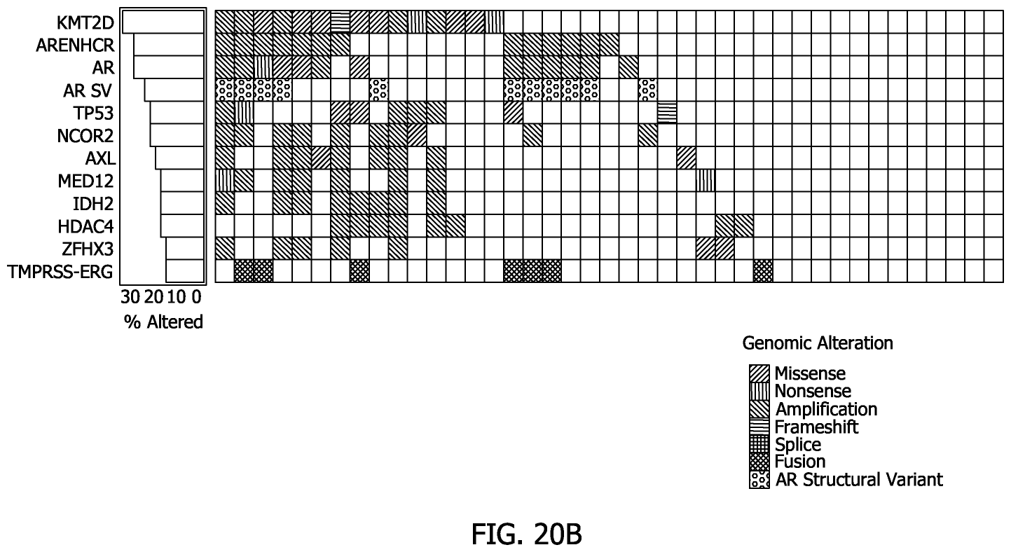

AR gene body or AR enhancer structural variations were identified in 50% of patients, as summarized in FIG. 17, FIG. 20A, and FIG. 20B. Structural variations, single nucleotide variants, insertions/deletions, and genomic rearrangements were also identified, with 70% of patients having at least one detectable alteration detected. Indicative of specificity, only 12% of healthy donors had any genomic alteration detected (one non-driver SNV detected in each of 3 cases), and 0% had any evidence of copy number alteration or genomic rearrangement (including no cases of AR/enhancer structural variation or TMPRSS2-ERG fusion in healthy donors). TMPRSS2-ERG fusions were detectable in 17% of patients, as illustrated in FIGS. 18 and 19.

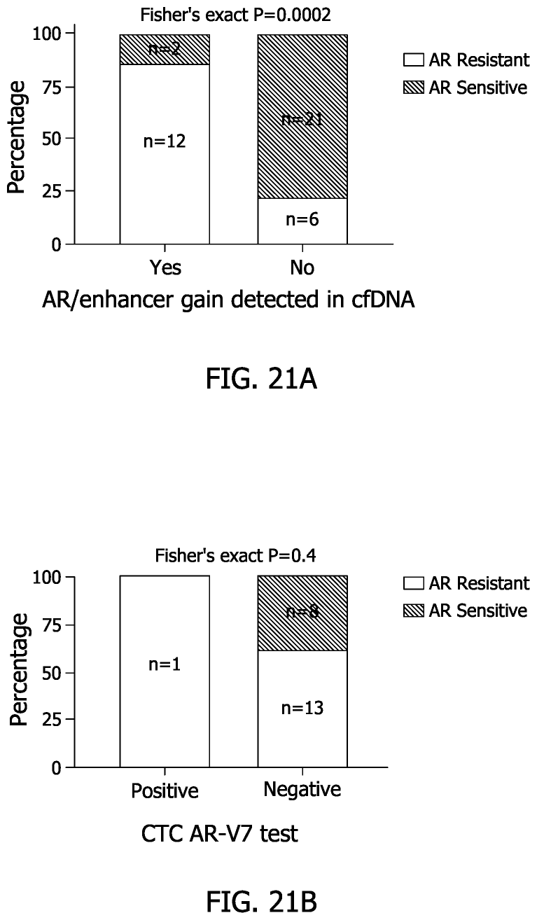

Table 5 is a summary of detected gains in copy number alterations targeting AR or AR enhancer (ARENHCR) among patients classified as resistant to AR-targeted therapies. As illustrated in FIG. 21A, detected gains in copy number alterations targeting AR or AR enhancer were significantly correlated with resistance to AR-directed therapies.

TABLE 5
Copy Number Alterations in mCRPC Patients

| sample | AR | ARENHCR | sample | AR | ARENHCR |
|---|---|---|---|---|---|
| PB046C1 | Gain | Gain | PB174C1 | Gain | Gain |
| PB087C1 | Gain | Neutral | PB183C1 | Gain | Gain |
| PB088C1 | Gain | Gain | PB204C1 | Gain | Gam |
| PB106C1 | Gain | Gain | PB226C1 | Neutral | Gain |
| PB132C1 | Gain | Gain | PB270C1 | Gain | Gain |
| PB140C1 | Gam | Gain | PB282C1 | Neutal | Gain |
| PB142C1 | Gain | Gam | PB314C1 | Neutral | Gain |

### Example 7: Comparison of EnhanceAR-Seq Cell-Free DNA Assay to CTC AR-V7 CTC Assay

To compare the efficacy of the EnhanceAR-Seq Cell-free DNA Assay to the corresponding efficacy of the standard-of-care CTC AR-V7 CTC assay, the following experiments were conducted.

The incidence of SVs identified by EnhanceAR-Seq as described in Ex. 6 was compared to the results of a CTC AR-V7 assay performed in parallel on a subset of the patient cohort described in Ex. 6. The commercial Genomic Health CTC AR-V7 assay was run at a similar timepoint as the EnhanceAR-Seq assay in 22 patients from the cohort. One patient had a positive AR-V7 result, and 21 patients had negative AR-V7 results. There was no correlation observed between structural variations in AR or its enhancer identified by EnhanceAR-Seq and the results from the CTC AR-V7 assay, as summarized in FIG. 21B.

### Example 8: Validation of EnhanceAR-Seq Cell-Free DNA Assay Using Tumor Biopsy Sequencing

To further validate the efficacy of EnhanceAR-Seq-based Cell-free DNA assay, the following experiments were conducted.

Fourteen patients from the cohort described in Ex. 6 consented to additional tissue-based analyses using biopsy samples. Among these, eight patient biopsy samples were obtained and analyzed by targeted gene sequencing using the Tempus platform, and six patient biopsy samples were obtained and analyzed using the EnhanceAR-Seq gene panel described in Ex. 6. Tissue sequencing revealed concordance between the tissue sequences and matched plasma sequences. Overall, structural variations in AR were highly concordant between tissue and plasma, while concordance for other genomic alterations was low. Notably, TMPRSS2-ERG fusions were not assessed using the Tempus panel. The high concordance of AR-based findings validated the efficacy of the AR-based analyses using the EnhanceAR-Seq gene panel as described in Ex. 6.

### Example 9: Clinical Analysis of EnhanceAR-Seq Cell-Free DNA Assay Using Tumor Biopsy Sequencing

To assess the clinical efficacy of the EnhanceAR-Seq Cell-free DNA assay, the following experiments were performed.

Clinical analyses similar to the analyses described in Ex. 4 and Ex. 5 were performed to associate the EnhanceAR-Seq gene panel results with clinical data from the cohort of patients described in Ex. 6. AR-related structural variations (AR SV) were associated with PSA progression-free survival (PFS; primary endpoint), as well as PSA response (secondary endpoint), correlation with AR resistance (secondary endpoint), and overall survival (OS; secondary endpoint), and were further compared to corresponding associations derived using the standard-of-care AR-V7 CTC assay performed in its clinically approved space as described in Ex. 8.

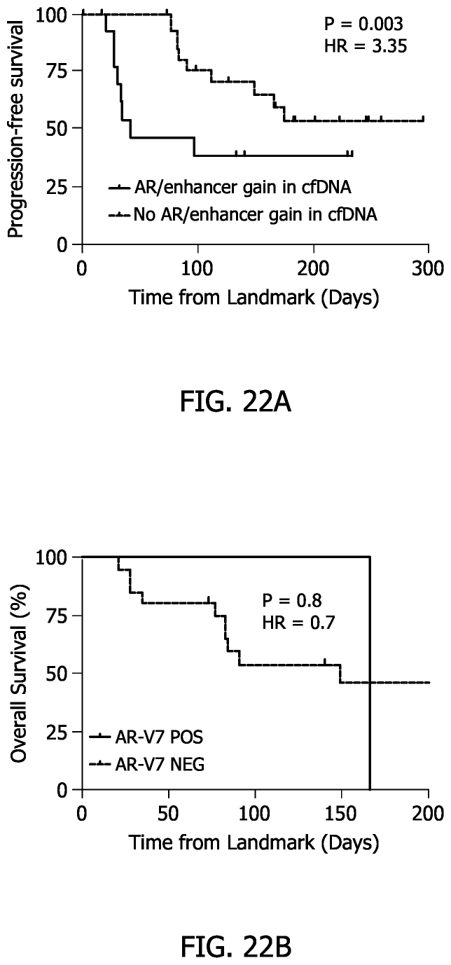

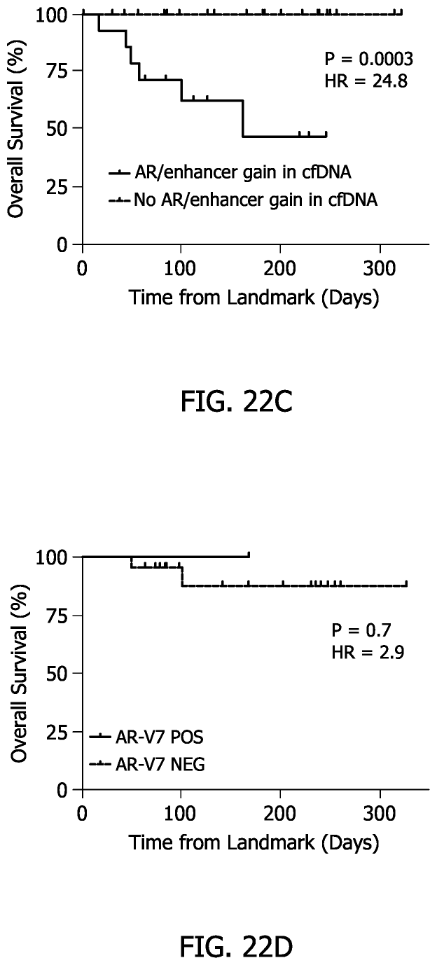

As summarized in FIG. 22A and FIG. 22C, patients with AR/AR enhancer structural variations had significantly worse PFS and OS than patients without AR/enhancer SVs (PFS at 60 days, 45% vs. 100%, OS at 180 days 45% vs. 100%, P<0.005). Notably, all 7 patients without AR/enhancer structural variations who developed disease progression on AR-directed therapy had evidence of secondary resistance rather than primary resistance; there were no deaths in this subset during follow-up similar to patients without detected AR/enhancer SVs. In contrast, the CTC AR-V7 assay was poorly predictive of PFS (FIG. 22B) and OS (FIG. 22D). The CTC AR-V7 assay was also poorly predictive of resistance to AR-directed therapy, identifying 0% of patients who developed resistance to AR-directed therapy, compared to ˜70% identified by EnhanceAR-Seq, and 100% with primary resistance identified by EnhanceAR-Seq.

A Cox proportional hazards model was used to estimate hazard ratios (HRs) as described in Ex. 4. Table 6 is a comparison of the results of the Cox proportional hazards regression for progression-free survival from the date of acquisition of the samples subjected to analysis using the EnhanceAR-Seq assay versus the CTC AR-V7.

TABLE 6
Cox proportional hazards regression for progression-free
survival from the sample landmark

| Parameter | P-value | Hazard Ratio |
|---|---|---|
| Age | NS | ~1 |
| Non-Caucasian Race | NS | ~1 |
| Line of Systemic Therapy | NS | ~1 |
| AR-V7 CIC Assay | 0.24 | 1.27 |
| AR/enhancer detected in cfDNA | 0.0325 | 3.37 |

* Sample landmark defined as date of blood sample acquisition; #P-value calculated by Likelihood ratio test, Hazard Ratio as exp(Beta); 1AR structural variation (SV) was measured in cell-free DNA (cfDNA) using EnhanceAR-Seq assay; 2AR-V7 splice variant measured in circulating tumor cells (CTCs) using Genomic Health/Epic Biosciences assay

Previously published results have indicated that amplification of the AR gene body was associated with shorter time-to-progression by univariate analysis, but not by multivariate analysis. To corroborate this earlier finding and to demonstrate the added benefit of tracking other structural variations in AR as well as amplifications in the AR enhancer, patients were stratified based on AR gene body amplification status, and PFS and OS were measured. PFS was slightly significant on univariate analysis, but not by multivariate analysis, and OS remained insignificant, suggesting the more comprehensive approach of cell-free DNA analysis implemented in EnhanceAR-Seq can detect high-risk disease more sensitively than examining AR gene body amplification alone.

The results of these experiments demonstrated that structural variation detection in the AR gene body or enhancer was strongly associated with resistance to AR-directed treatment and was associated with significantly worse clinical outcomes. The EnhanceAR-Seq assay significantly outperformed the CTC AR-V7 assay.

### Example 10: Monitoring of Serial Samples Using EnhanceAR-Seq Cell-Free DNA Assay

To assess the efficacy of serial sampling of plasma samples using the EnhanceAR-Seq Cell-free DNA assay, the following experiments were performed.

Serial samples were obtained from a subset of the patient cohort described in Ex. 6 during systemic treatment and these serial samples were analyzed using the EnhanceAR-Seq assay as described in Ex. 6.

Seven patients from the cohort described in Ex. 6 received serial blood draws every ˜3 months. All samples were analyzed using the EnhanceAR-Seq assay described in Ex. 6. Four of these patients had AR/AR enhancer structural variations detected at baseline and three did not. All four patients with AR/enhancer SVs detected at baseline developed primary therapeutic resistance, and AR/enhancer SVs were detected along with other genomic alterations at subsequent serial timepoints, indicating intra-patient assay concordance. Among the remaining three patients, one patient developed secondary AR resistance to abiraterone and the other two remained sensitive to AR-directed treatment. In the patient who developed secondary AR resistance, amplification of the upstream AR enhancer was detected at the subsequent two serial timepoints.

Five patients also underwent serial testing using the AR-V7 assay which was correlated with clinical outcomes. One patient was initially negative and subsequently became positive, correlating with secondary resistance to AR-directed therapy. Kaplan-Meier analysis for PFS was performed accounting for serial sampling, comparing AR/enhancer structural variation by cell-free analysis to AR-V7 splice variant by CTC analysis (alteration ever detected vs. never detected). Log-rank P value based on alteration status remained highly significant based on cell-free DNA AR/enhancer detection with slightly higher hazard ratio than baseline analysis alone, but remained insignificant based on CTC AR-V7 status.

### Example 11: Predicting Resistance to AR-Directed Therapy in Patients with Metastatic Prostate Cancer

Study Design and Methods

40 patients with metastatic prostate cancer treated with at least 1 month of standard-of-care AR-directed treatment (e.g., abiraterone or enzalutamide) were enrolled in a study designed to assess whether resistance to AR-directed therapy in patients with metastatic prostate cancer could be predicted by tracking genomic alterations in the AR enhancer in addition to the AR gene body (AR/enhancer) in plasma cell-free DNA (cfDNA). All patients were maintained on standard androgen deprivation therapy (i.e., luteinizing hormone-releasing hormone receptor agonist or antagonist). Prior treatment with other systemic agents, including chemotherapy, was allowed. Patients with evidence of any active nonprostate malignancy other than localized skin cancer were excluded from the study.

Eligible patients underwent blood collection for cfDNA analysis at the time of enrollment. Between 10 and 20 mL of peripheral blood was collected in K2EDTA Vacutainer tubes (Becton Dickinson). Tubes were centrifuged at 1,200 g for 10 minutes, then plasma separated and centrifuged for another 5 minutes at 1,800 g. Plasma was then frozen at −80° C. prior to cfDNA processing and analysis. Leukocyte-enriched plasma-depleted whole blood (PDWB) was also collected and frozen at −80° C. for isolation of germline genomic DNA.

cfDNA was extracted from plasma using the QiaAmp Circulating Nucleic Acid Kit (Qiagen) according to the manufacturer's instructions. cfDNA concentration was measured with a Qubit 4.0 Fluorometer (Thermo Fisher Scientific) using the dsDNA High Sensitivity Assay Kit (Thermo Fischer Scientific). cfDNA fragment size was determined using an Agilent 2100 Bioanalyzer with the High Sensitivity DNA Kit (Agilent Technologies). A median of 32 ng was inputted into sequencing library preparation based on the percentage of cfDNA in the 70-450 bp region of the bioanalyzer electropherogram. The QIAamp DNA Micro Kit (Qiagen) was employed to extract genomic DNA from 100 ul of PDWB. Genomic DNA from PDWB was fragmented prior to library preparation using a LE220 focused ultrasonicator (Covaris).

A targeted sequencing assay of plasma cfDNA (EnhanceAR-Seq) was developed to monitor genomic alterations in the AR gene and AR enhancer loci and other frequently altered genes in in metastatic prostate cancer. To develop the assay, a hybrid-capture gene panel was designed to target the complete AR genebody (including introns), 30 kb of the AR enhancer, and exons of 84 other genes that have been shown to harbor genomic alterations in mCRPC. To gain finer detail for copy number analysis in the full AR/enhancer locus, 500 bp targeted regions were evenly placed (1 kb apart) between 500 kb upstream of the AR enhancer and 500 kb downstream of the AR gene body. The panel also included a TMPRSS2-ERG gene fusion hotspot intronic region (13 kb) in the TMPRSS2 gene to detect a subset of TMPRSS2-ERG gene fusions. Additionally, 12 genes least frequently affected by copy number alteration in mCRPC (surveyed in prior WGS data5) were included in the panel as controls for copy number analysis, and three genes included to assess clonal hematopoiesis. NimbleDesign (Roche) was used to convert the gene panel into a SeaCap EZ Prime Choice probe set (Roche).

The genes included in the EnhanceAR-Seq sequencing panel are shown below in Table 7, with copy number and clonal hematopoiesis control genes listed in the right-most column.

TABLE 7
Genes included in the EnhanceAR-Seq Targeted Sequencing Panel

| | | | | | | |
|---|---|---|---|---|---|---|
| AKT1 | CDK4 | ETV5 | KDM6A | NFE2L2 | SPOP | CYP4F3 |
| AKT2 | CDK6 | FANCA | KMT2C | NKX3-1 | TMPRSS2 | ELF4 |
| AKT3 | CDKN1B | FANCC | KMT2D | PIK3CA | TP53 | SLITRK2b |
| APC | CDKN2A | FANCD2 | KRAS | PIK3CB | ZBTB16 | SPANXN1 |
| AR | CHD1 | FANCE | MDM2 | PIK3R1 | ZFHX3 | SPTY2D1 |
| AR Enhancer | CLU | FANCF | MDM4 | PMS1 | ZNRF3 | TPTE |
| ARID1A | CTNNB1 | FANCG | MED12 | PMS2 | | TRIM43 |
| ASXL2 | CUL1 | FBXW7 | MET | PRKDC | | ACTRIB |
| ATM | ERCC1 | FOXA1 | MLH1 | PTEN | | AKAP7 |
| ATR | ERCC2 | FOXP1 | MSH2 | RAD51B | | ANKRD36 |
| AXL | ERCC3 | GNAS | MSH3 | RAD51C | | APLN |
| BRAF | ERCC4 | HDAC4 | MSH6 | RB1 | | CYP4F22 |
| BRCA1 | ERCC5 | HRAS | MYC | RNF43 | | ASXL1 |
| BRCA2 | ERG | HSD3B1 | NCOA2 | RUNX1 | | DNMT3A |
| CCND1 | ETV1 | IDH1 | NCOR1 | RYBP | | TET2 |
| CDK12 | ETV4 | IDH2 | NCOR2 | SMARCA1 | | |

cfDNA and PDWB DNA library preparation using a workflow was performed with duplex barcoded adapters. Next generation sequencing (NGS) was then performed on an Illumina HiSeq4000 with 2×150 bp paired-end reads, with 12 samples sequenced per lane, dedicating ˜60 million reads per sample. A custom bioinformatics pipeline was then applied.

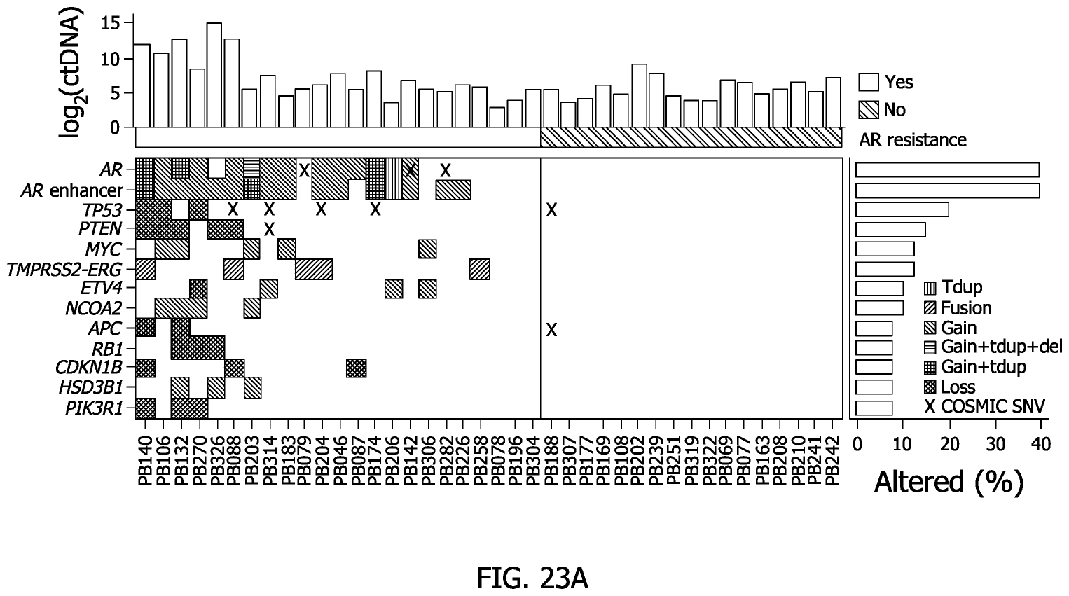

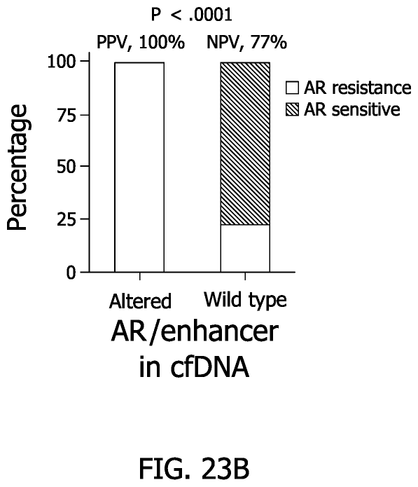

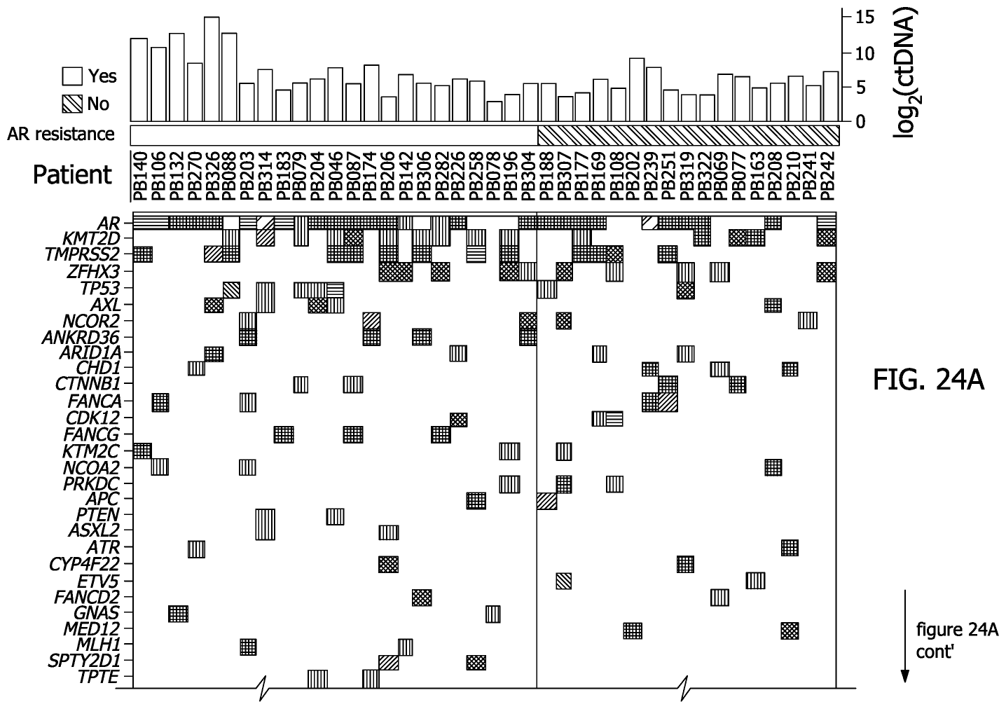

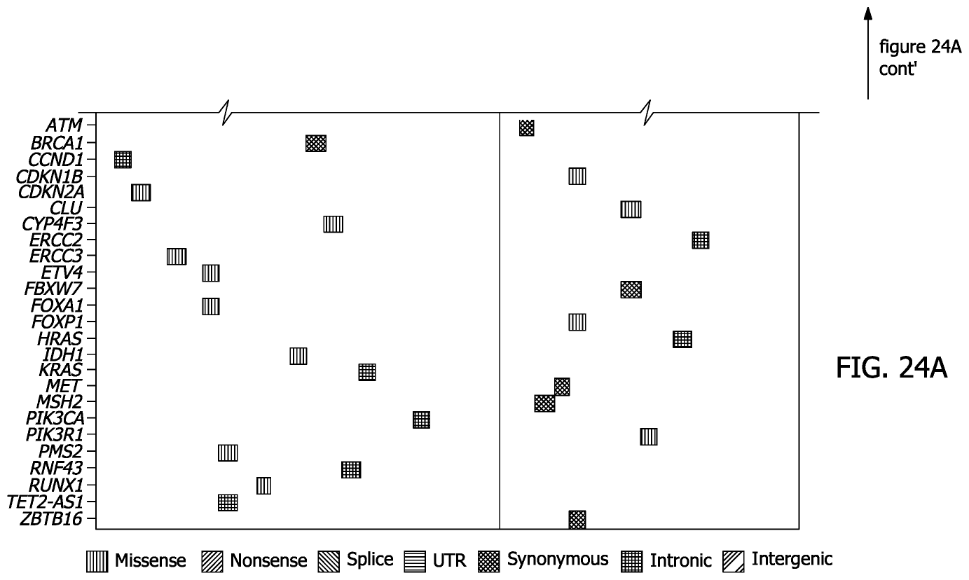

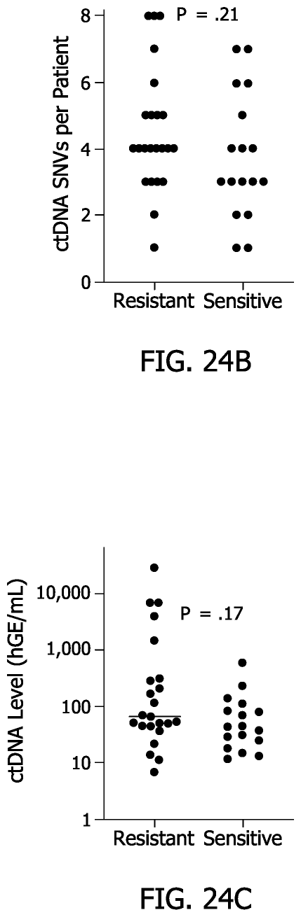

Cell-free (cfDNA) sequencing results were analyzed for single nucleotide variants (SNVs) and insertions/deletions (indels) using the EnhanceAR-Seq bioinformatic pipeline. cfDNA sequencing reads were de-multiplexed using sample-level index barcodes, mapped to the human reference genome, filtered for properly paired reads, filtered for bases with Phred quality score ≥30, then de-duplicated using unique molecular identifiers. Background-polishing using 12 healthy donor plasma samples was performed to reduce stereotypical base substitution errors using an integrated digital error suppression (iDES) method. Variant-calling using the EnhanceAR-Seq pipeline was then performed to call SNVs and indels from patient plasma using matched plasma-depleted whole blood (PDWB) as the background reference, filtered further to remove potential SNPs with variant allele fraction (vAF) >45%, loci with de-duplicated depth <100, and mutations in the canonical clonal hematopoiesis genes ASXL1, DNMT3A and TET26-8. Nonsynonymous SNVs and indels ≥2 base pairs in plasma, not present in matched PDWB, not present in the Genome Aggregation Database (gnomAD)10 at a >0.0001 frequency, and indexed in the Catalogue of Somatic Mutations in Cancer (COSMIC)11 were reported in a final dataset shown in FIGS. 23A and 23B. Mutations in AR that met these criteria were considered positive by EnhanceAR-Seq. An additional SNV analysis using the filters described above but not requiring COSMIC indexing was performed to measure overall ctDNA SNV burden (number of SNVs detected per patient) and levels (based on mean vAF and cfDNA concentration), as shown in FIGS. 24A, 24B and 24C.

Cell-free DNA sequencing results were de-multiplexed using sample-level index barcodes, mapped to the human reference genome, then de-duplicated using Picard based on identical start/end coordinates. Copy number analysis was performed based on a read depth approach. First, the genome was binned (larger bins for non-targeted regions and smaller bins for targeted regions) and read depth ratios for bins between plasma cfDNA and matched PDWB control samples were calculated and corrected for biases in GC content, sequence repeats, and target density using CNVkit. Subsequently, read depth ratios were centralized by subtracting the mean log 2 ratios of all bins across chromosomes and normalized using read depth ratios from bins overlapping with copy number control genes. Copy number segmentation was performed using DNACopy. To obtain the background read depth ratio for individual genes, the same analysis was performed on 24 pairs of plasma and matched PDWB control cfDNA samples from male healthy donors. Finally, a gain (or loss) event in patient plasma was called when the calculated log 2 ratio was four standard deviations above (or below) the median log 2 ratio of that locus in healthy plasma. Genes whose log 2 ratios showed high variability or deviation from zero in healthy plasma samples (median >0.2 or standard deviation >0.2) were excluded from copy number analysis.

The targeted panel was designed to capture structural variation (SV) breakpoints targeting full-length AR (including intronic regions) and the TMPRSS2-ERG fusion hotspot in an intron of TMPRSS2. SVs including tandem duplications were called using Lumpy and Manta using plasma samples with matched PDWB control samples. Subsequently, SVs with breakpoints overlapping the blacklist and low complexity regions or those with both breakpoints falling in non-targeted regions were removed. Additional filtering was applied to retain only SVs with at least 2 supporting discordant read pairs or split reads and with high confidence regarding breakpoint positions (based on the width of the confidence interval provided by Manta or Lumpy being <5 bases), and filtering out SVs with abnormally high read support (>150 discordant read pairs or split reads) in patient plasma cfDNA.

Clinical Outcomes

The primary clinical endpoint was primary or secondary resistance to AR-directed therapy. Primary resistance was defined as prostate-specific antigen (PSA) progression, change of therapy or death within 4 months of treatment initiation, or radiographic progression within 6 months. Secondary resistance was defined as PSA progression, change of therapy, radiographic progression or death outside of this timeframe. PSA progression was defined as an increase of ≥25% above nadir and ≥2 ng per milliliter, with confirmation ≥3 weeks later (PCWG3). Secondary endpoints were progression-free survival (PFS) defined as the time to PSA progression by PCWG3 criteria or death, or last known date of PSA measurement in non-progressors, and overall survival (OS) defined as time to death or to last follow up for alive patients. PFS and OS were calculated from time of study enrollment.

Results

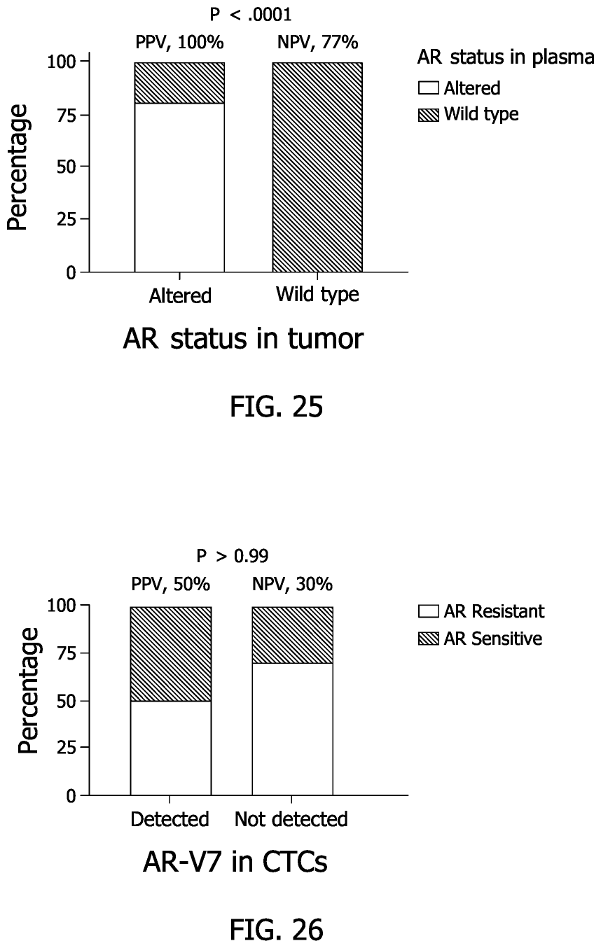

The most frequent genomic events detected in plasma cfDNA were AR/enhancer alterations (most commonly copy number gain and tandem duplication) present in 18 patients (45%), including a 40% amplification rate in the AR enhancer region, as shown in FIG. 23A. Three patients (8%) were found to have independent AR enhancer amplification without AR gene body amplification, consistent with previous tissue-based results (see FIG. 23A). Other genes frequently found in cfDNA to be targeted by alterations included TP53 and PTEN which demonstrated copy number loss in 6 patients (15%) and COSMIC-annotated nonsynonymous single nucleotide variants in 5 cases (13%) (See FIG. 23A). TMPRSS2-ERG gene fusion was also detected in 5 cases (13%) (see FIG. 23A). Overall, genomic alterations in AR were 80% concordant between tissue and plasma, as shown in FIG. 25.

AR/Enhancer Alterations in cfDNA Associated with Clinical Resistance

The greatest concordance was observed between genomic events and clinical resistance to AR-directed therapy for alterations in the AR locus including the enhancer (see FIGS. 23A-C). Alterations in the AR/enhancer locus predicted resistance with 78% sensitivity and 100% specificity (see FIG. 23B). There was a highly significant correlation between alterations detected in AR/enhancer in cfDNA and resistance to AR-directed therapy (P<0.0001). All three cases with AR enhancer amplification in cfDNA in the absence of AR gene body amplification progressed to resistance at a median of 5.3 months (range 0.6-8.0), indicative of improved sensitivity in identifying resistance when tracking the AR enhancer in addition to the gene body. The AR-V7 Nucleus Detect CTC assay was run at a median of 16 days from cfDNA analysis in 25 patients, including within 24 hours of cfDNA testing for 10 patients. AR-V7 was detected in CTCs from 2 patients (8%) and negative in the remaining 23, as shown in FIG. 26.

AR/Enhancer Alterations in cfDNA Portended Poor Progression-Free Survival (PFS)

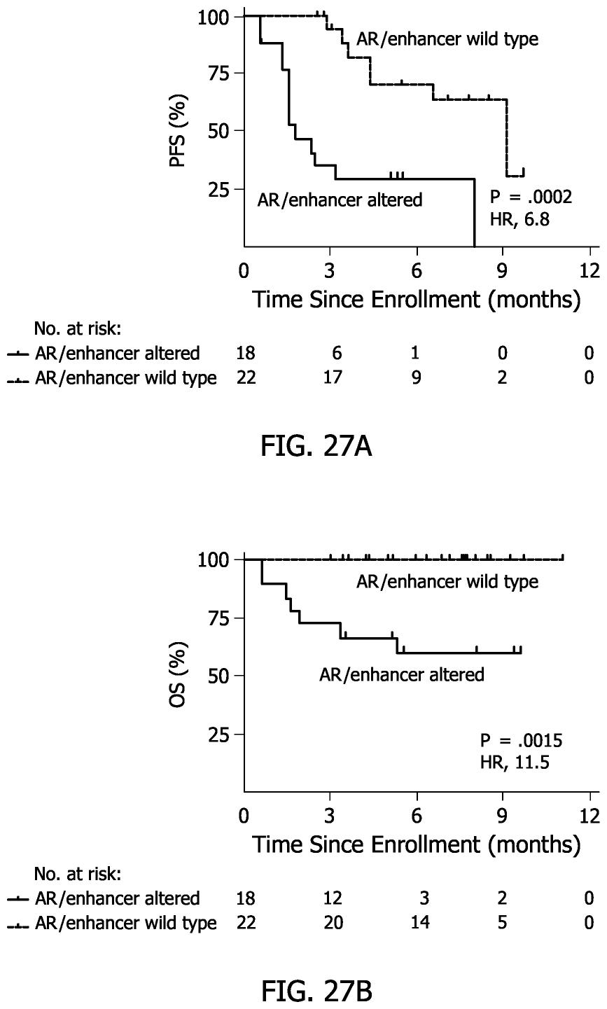

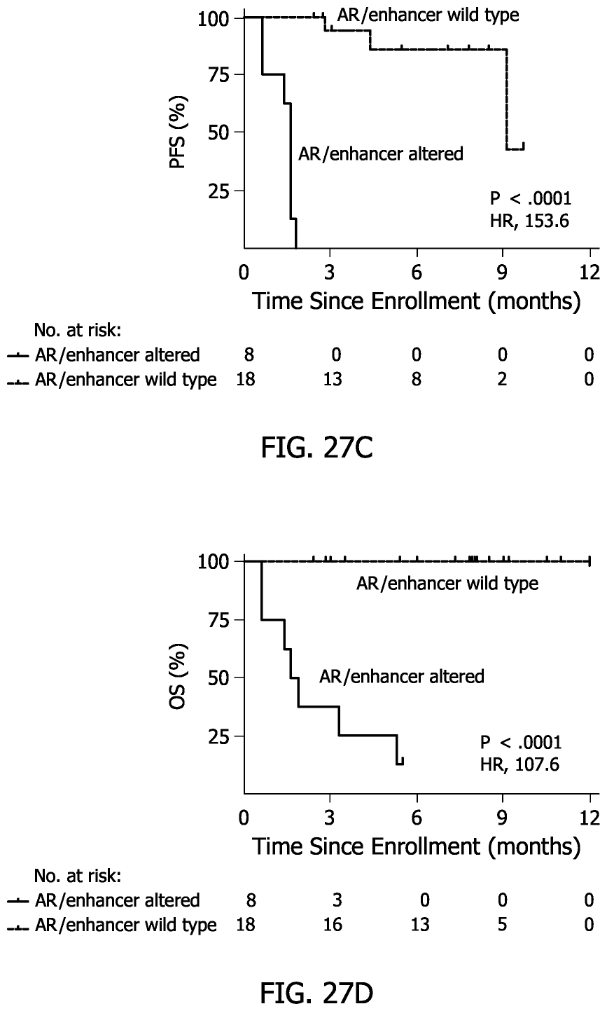

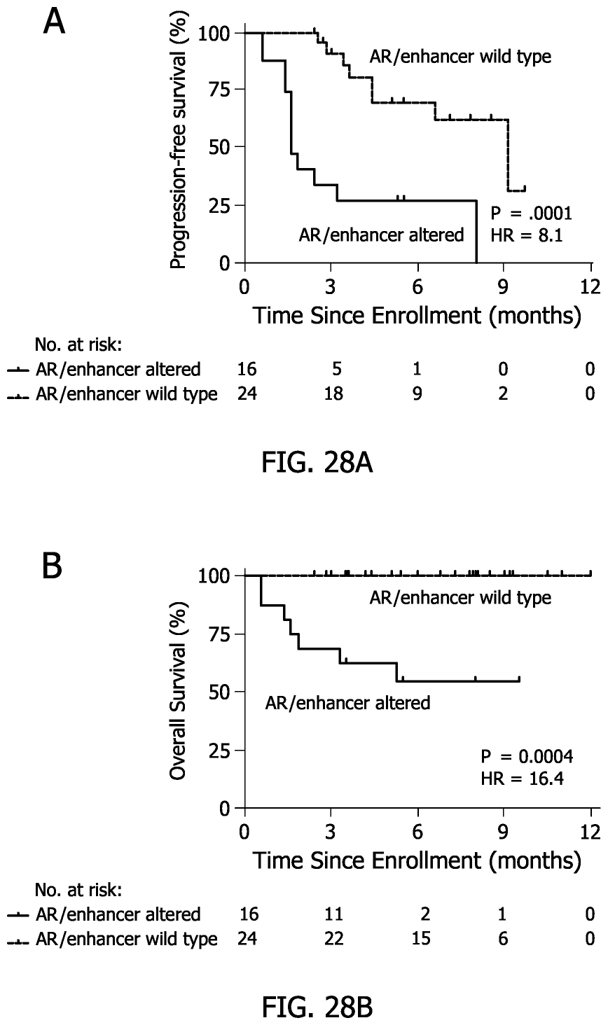

PFS was significantly shorter among men with detected AR/enhancer alterations in plasma cfDNA (18 patients, 45%) compared to those without (22 patients, 55%) (HR 6.8; 95% CI, 2.5-18.6; P=0.0002), as shown in FIG. 27A. PFS remained significantly shorter with similar hazard ratio when restricting the analysis to just the AR enhancer region (HR 8.1; 95% CI 2.8-23.6; P=0.0001), as shown in FIG. 28A. cfDNA-detected alterations in the AR/enhancer locus or the AR enhancer alone remained highly significant by multivariate Cox proportional hazards regression, which included important baseline characteristics such as PSA concentration, circulating tumor DNA (ctDNA) level, number of lines of therapy received in the metastatic setting, prior enzalutamide vs. abiraterone treatment, metastatic disease burden and time since diagnosis. It was also found that overall ctDNA levels and mutational burden did not correlate with clinical outcomes, nor were they significantly different between patients who developed AR-resistance vs. remained AR-sensitive, as shown in FIGS. 24A-C.

AR/Enhancer Alterations in cfDNA Portended Poor Overall Survival (OS)

Although median follow-up of the cohort from time of enrollment was only 6.0 months, a preliminary OS analysis was performed. OS was significantly shorter among men with detected AR/enhancer alterations in plasma cfDNA compared to those without (HR 11.5; 95% CI 2.5-52.1; P=0.0015) (FIG. 27B). OS remained significantly shorter with a high hazard ratio when ignoring AR gene body alterations and restricting the analysis to just the AR enhancer region (HR 16.4; 95% CI 3.5-77.2; P=0.0004) (see FIG. 28B).

AR/Enhancer Alterations in cfDNA in Primary Versus Secondary Resistance

The cohort included nine primary resistant and 14 secondary resistant cases. In all cases of primary resistance, patients experienced no response, while in cases of secondary resistance, patients experienced a temporary treatment response before ultimately progressing on AR-directed therapy. Notably, the previously published AR-V7 assay had only been shown to be capable of identifying primary resistance, albeit with limited sensitivity. When EnhanceAR-Seq was utilized, positive predictive value of cfDNA-derived AR/enhancer alterations for primary resistance was 100%, with every positive case progressing within 3 months and all but one dying within 6 months of study enrollment (see FIGS. 27C and 27D). The sensitivity of the assay for detecting primary resistance was 89%, higher than the 71% observed for secondary resistance, while specificity remained 100%.

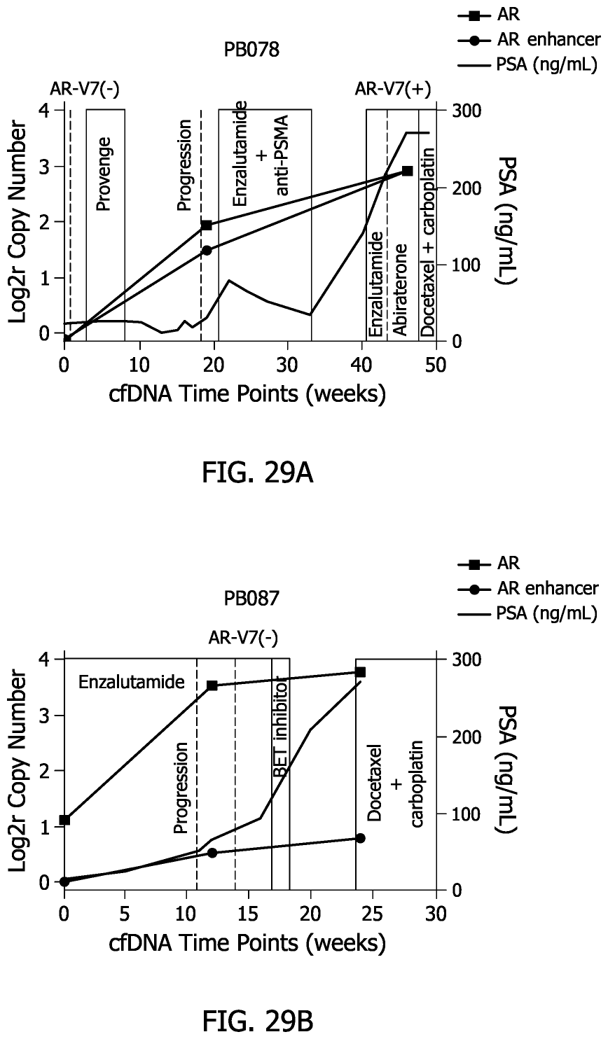

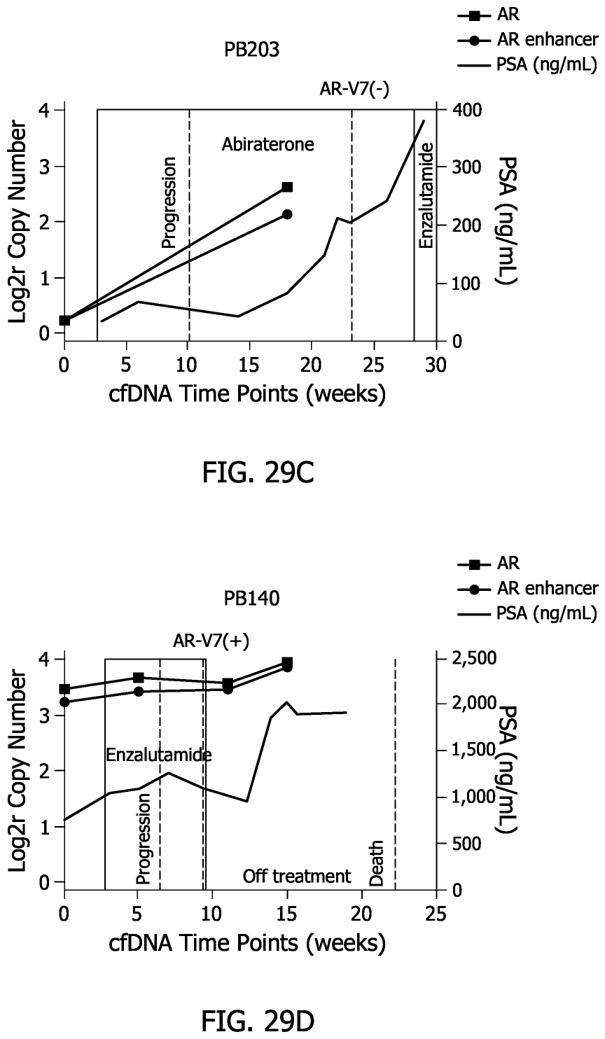

Serial samples in four patients were obtained with at least one timepoint being during AR-directed therapy, as shown in FIGS. 29A-D. For patient PB078 (FIG. 29A), EnhanceAR-Seq detected no evidence of AR/enhancer alterations at enrollment, and AR-V7 detection in CTCs was also negative. At 19 and ˜45 weeks later, EnhanceAR-Seq revealed significantly elevated copy number amplification of both the AR gene body and enhancer, while the patient was actively developing resistance to enzalutamide followed by abiraterone. The CTC AR-V7 assay also became positive at ˜45-weeks. Patients PB087 and PB203 similarly showed rapid increases in AR/enhancer copy number on enzalutamide and abiraterone, respectively, while AR-V7 testing remained negative (FIGS. 29B and 29C). Cell-free AR/enhancer amplification preceded rise in PSA and clinician-recognized resistance leading to therapy change. For patient PB140 (FIG. 29D), AR/enhancer copy numbers increased more subtly on serial analysis, however in this case the baseline copy numbers for AR and its enhancer were already >8-fold elevated; reflective of this, the patient progressed rapidly 6 weeks after study enrollment and died from mCRPC at 22 weeks. These data demonstrate the value of using cell-free DNA based AR/enhancer analysis as a precision modality to monitor treatment resistance in metastatic prostate cancer patients undergoing AR-directed therapy. These data additionally support the potential value of serial timepoint analysis, especially in the secondary resistance setting where AR/enhancer amplification may not be apparent at baseline. These data further show that the assay may be used to inform clinicians when to trial a different AR-directed treatment (when AR/enhancer copy numbers remain low) or switch to a different therapy-type altogether (when AR/enhancer copy numbers have risen high)

At the conclusion of the study, it was shown that EnhanceAR-Seq outperformed the CTC AR-V7 test utilized clinically (compare FIG. 26 with FIG. 23B). Within the study cohort, nearly every patient with detectable alterations in AR or its enhancer in cell-free DNA developed resistance and progressed despite a relatively short follow-up period. AR/enhancer alterations were associated with significantly worse PFS and OS. In contrast, the Genomic Health CTC AR-V7 assay was positive in only 8% of tested cases and did not correlate significantly with outcomes.

### Example 12: Cell-Free Alterations in the AR/Enhancer Locus Measured Before AR Signaling Inhibition Portend Poor Overall Survival in Metastatic Castration Resistant Prostate Cancer Patients

A study was conducted to determine whether AR/enhancer genomic alterations detected in plasma cell-free DNA prior to the administration of first-line AR-selective inhibitors (ARSIs) can predict survival in metastatic castration resistant prostate cancer (mCRPC) patients. EnhanceAR-Seq was applied to plasma cell-free DNA isolated from 20 mCRPC patients. Assay results were correlated with patient overall survival (OS) and progression-free survival (PFS) from the time of blood collection.

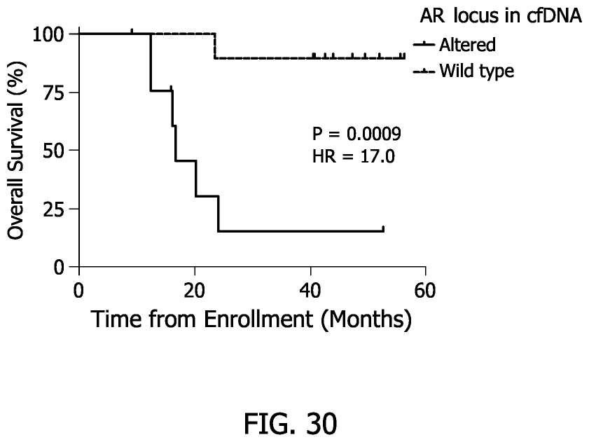

Median follow up time was 32 months. Seventeen patients had blood plasma analyzed before first-line ARSI treatment, while three patients had received prior ARSI treatment before blood collection. EnhanceAR-Seq revealed that the most frequent genomic events detected were AR/enhancer alterations (copy number gain, tandem duplication or missense mutations) in 9 patients (45%), of which 5 patients had both AR gene body and enhancer copy number gain. The other 4 patients each had a single genomic event detected by EnhanceAR-Seq: AR amplification, AR enhancer amplification, AR and AR enhancer tandem duplication, and AR W742C single nucleotide variation. Cell-free DNA-detected alterations in the full AR locus including the AR enhancer were highly significant for inferior OS (P=0.0009; HR=17.0) (see FIG. 30B) but not for PFS (P=0.2; HR=2.2) by Kaplan-Meier analysis across all 20 patients. Subset analysis of the 17 patients with plasma analyzed prior to first-line ARSI treatment revealed that AR/enhancer alterations predicted significantly worse OS with a median survival of 16.1 months vs. not-reached (P=0.0009; HR=14.1). As such, AR locus alterations detected by EnhanceAR-seq in plasma cell-free DNA collected prior to ARSI administration correlated with significantly worse overall survival in patients with mCRPC.

### Example 13: A Unified Pipeline to Detect Small Mutations, Structural Variations, and Copy Number Alterations from Targeted Cell-Free DNA Sequencing in Cancer

A study was conducted to develop an integrated bioinformatics pipeline for SV and copy number alteration (CNA) detection of cfDNA following targeted hybrid-capture next-generation sequencing (NGS), along with standard SNV and indel analysis.

SVs were first detected using Manta, Lumpy, and Delly in plasma cfDNA in comparison with matched peripheral blood leukocyte (PBL) DNA samples from cancer patients, then combined to identify consensus SVs and genotyped throughout samples from patients and healthy individuals. Next, consensus SVs were called somatic events if they were supported by split reads and discordant read pairs in cfDNA samples from patients but not in matched PBL or healthy donor cfDNA samples. For CNA analysis, the ratio of read depth between patient-derived plasma cfDNA and a panel of healthy controls was calculated across genomic bins using a CNVkit tool, followed by bias correction and recentralization using CNA negative control genes to account for read coverage imbalances in targeted NGS. Lastly, SNV and indel analysis was integrated from the EnhanceAR-Seq pipeline.

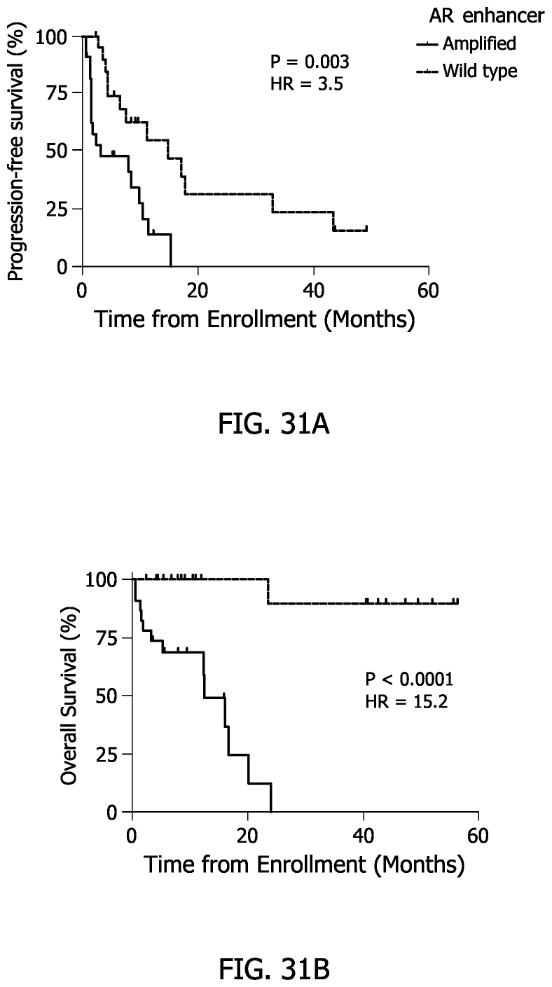

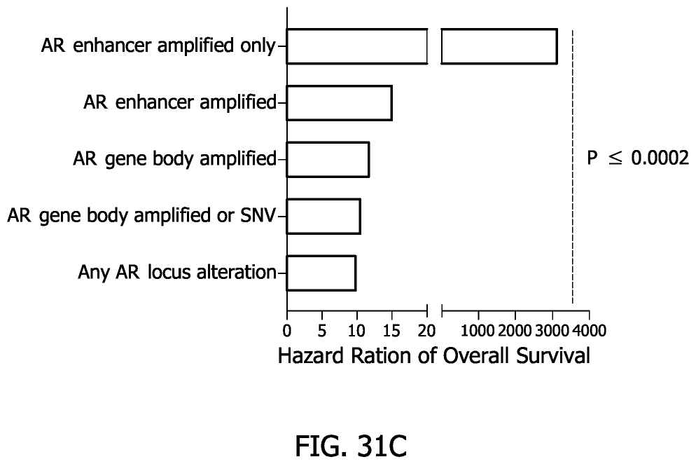

The study pipeline was applied to targeted hybrid-capture NGS data from 48 patients across two independent cohorts of metastatic castration resistant prostate cancer (mCRPC). The targeted panel covered the full-length AR gene body and a hotspot region of TMPRSS2-ERG fusion break points. Consistent with earlier whole genome studies, known CNAs and SVs in tumor suppressors, oncogenes and regulatory elements including AR gene and AR enhancer duplications (22/48, 46% of patients), TMPRSS2-ERG gene fusions (9/48, 19%), PTEN and TP53 loss (8/48, 17%) were confirmed. Notably, the pipeline outperformed FACTERA which did not detect any TMPRSS2-ERG gene fusions or AR/enhancer tandem duplications. Subsequent analysis showed high concordance between plasma cfDNA and matched tumor biopsies, and the pipeline recapitulated the landscape of SVs and CNAs in an in silico cfDNA simulation from tumor biopsies. Finally, it was shown that alterations of the AR/enhancer locus detected by the pipeline were strongly associated with treatment resistance, patient progression-free and overall survival in mCRPC (see FIGS. 31A-C).

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A method for identifying a prostate cancer treatment for a subject, the method comprising:
obtaining a fluid sample from the subject, the fluid sample comprising noncellular DNA (ncDNA) from the subject;
transforming the ncDNA into a plurality of genomic alterations to determine if the ncDNA contains castration-resistant structural variations including at least one of an amplification or structural variation of an AR encoding an androgen receptor and an amplification or structural variation in an AR enhancer; and
identifying the prostate cancer treatment for the subject based on the plurality of genomic variations.

2. The method of claim 1, wherein identifying the prostate cancer treatment for the subject based on the plurality of genomic variations comprises:
identifying a non-AR focused treatment if the plurality of genomic variations includes at least one of the castration-resistant structural variations; and
identifying an AR-focused treatment if the plurality of genomic variations does not include at least one of the castration-resistant structural variations.

3. The method of claim 2, wherein:
the non-AR focused treatment comprises one or more of immunotherapy, radiotherapy, targeted therapy, and chemotherapy; and
the AR focused treatment comprises one or more AR-directed drugs, the AR-directed drugs comprising Abiraterone and Enzalutamide.

4. The method of claim 1, further comprising:
determining if the plurality of genomic variations contains DNA-repair deficiency-related structural variations including at least one of CDK12 mutations with tandem duplications, TP53 inactivation with inverted rearrangements and chromothripsis, and BRCA2 inactivation with deletions; and
identifying a DNA damage repair treatment if the plurality of genomic variations includes at least one of the DNA-repair deficiency-related structural variations, wherein the DNA damage repair treatment comprises a poly (ADP-ribose) polymerase (PARP) inhibitor.

5. The method of claim 1, wherein the fluid sample comprises at least one of a blood sample, a plasma sample, a urine sample, and a saliva sample.

6. The method of claim 1, wherein the transforming comprises contacting the fluid sample with one or more probes, each comprising a gene sequence selected from a gene panel, wherein the one or more probes are configured to capture the castration-resistant structural variations in the fluid sample.

7. The method of claim 6, wherein the gene panel comprises a copy number control gene and a clonal hematopoiesis gene.

8. The method of claim 7, wherein the gene panel comprises one or more genes selected from the group consisting of AKT1, AKT2, AKT3, APC, AR, AR Enhancer, ARID1A, ASXL2, ATM, ATR, AXL, BRAF, BRCA1, BRCA2, CCND1, CDK12, CDK4, CDK6, CDKN1B, CDKN2A, CHD1, CLU, CTNNB1, CUL1, ERCC1, ERCC2, ERCC3, ERCC4, ERCC5, ERG, ETV1, ETV4, ETV5, FANCA, FANCC, FANCD2, FANCE, FANCF, FANCG, FBXW7, FOXA1, FOXP1, GNAS, HDAC4, HRAS, HSD3B1, IDH1, IDH2, KDM6A, KMT2C, KMT2D, KRAS, MDM2, MDM4, MED12, MET, MLH1, MSH2, MSH3, MSH6, MYC, NCOA2, NCOR1, NCOR2, NFE2L2, NKX3-1, PIK3CA, PIK3CB, PIK3R1, PMS1, PMS2, PRKDC, PTEN, RAD51B, RAD51C, RB1, RNF43, RUNX1, RYBP, SMARCA1, SPOP, TMPRSS2, TP53, ZBTB16, ZFHX3, ZNRF3, CYP4F3, ELF4, SLITRK2b, SPANXN1, SPTY2D1, TPTE, TRIM43, ACTRIB, AKAP7, ANKRD36, APLN, CYP4F22, ASXL1, DNMT3A and TET2.

9. The method of claim 1, wherein the genomic variations further include additional structural variations, copy number alterations, tandem duplications, fusions, rearrangements, single nucleotide variants, insertions/deletions, and combinations thereof.

10. An assay kit comprising:
an assay comprising:
a probe set comprising a plurality of probes,
wherein the probe set is configured to transform a fluid sample comprising ncDNA into a plurality of genomic variations, wherein the genomic variations comprise at least one of an amplification or structural variation of an AR encoding an androgen receptor and an amplification or structural variation in an AR enhancer.

11. The kit of claim 10, wherein each probe of the plurality of probes comprises a gene sequence selected from a gene panel, wherein the plurality of probes are configured to capture the plurality of castration-resistant structural variations in the fluid sample.

12. The kit of claim 11, wherein the gene panel comprises a copy number control gene and a clonal hematopoiesis gene.

13. The kit of claim 11, wherein the gene panel comprises one or more genes selected from the group consisting of AKT1, AKT2, AKT3, APC, AR, AR Enhancer, ARID1A, ASXL2, ATM, ATR, AXL, BRAF, BRCA1, BRCA2, CCND1, CDK12, CDK4, CDK6, CDKN1B, CDKN2A, CHD1, CLU, CTNNB1, CUL1, ERCC1, ERCC2, ERCC3, ERCC4, ERCC5, ERG, ETV1, ETV4, ETV5, FANCA, FANCC, FANCD2, FANCE, FANCF, FANCG, FBXW7, FOXA1, FOXP1, GNAS, HDAC4, HRAS, HSD3B1, IDH1, IDH2, KDM6A, KMT2C, KMT2D, KRAS, MDM2, MDM4, MED12, MET, MLH1, MSH2, MSH3, MSH6, MYC, NCOA2, NCOR1, NCOR2, NFE2L2, NKX3-1, PIK3CA, PIK3CB, PIK3R1, PMS1, PMS2, PRKDC, PTEN, RAD51B, RAD51C, RB1, RNF43, RUNX1, RYBP, SMARCA1, SPOP, TMPRSS2, TP53, ZBTB16, ZFHX3, ZNRF3, CYP4F3, ELF4, SLITRK2b, SPANXN1, SPTY2D1, TPTE, TRIM43, ACTRIB, AKAP7, ANKRD36, APLN, CYP4F22, ASXL1, DNMT3A and TET2.

14. The kit of claim 10, wherein each probe of the plurality of probes are labeled with a radioactive or non-radioactive label.

15. The kit of claim 10, wherein the plurality of genomic variations further comprise additional copy number alterations, tandem duplications, fusions, rearrangements, single nucleotide variants, insertions/deletions and combinations thereof.

16. The kit of claim 10, wherein the plurality of genomic variations further comprises DNA-repair deficiency-related structural variations including at least one of CDK12 mutations with tandem duplications, TP53 inactivation with inverted rearrangements and chromothripsis, and BRCA2 inactivation with deletions.

17. The kit of claim 15, wherein the assay is configured to detect the plurality of genomic variations with a sensitivity of greater than about 20%.

18. A method for treating prostate cancer, the method comprising:
obtaining a fluid sample from a subject, the fluid sample comprising noncellular DNA (ncDNA) from the subject;
transforming the ncDNA into a plurality of genomic variations to determine if the ncDNA contains castration-resistant genomic variations including at least one of an amplification, structural variation, tandem duplication, or single nucleotide variation of an AR encoding an androgen receptor and an amplification, structural variation or tandem duplication in an AR enhancer; and
administering a prostate cancer treatment to the subject based on the plurality of genomic variations.

19. The method of claim 18, wherein the method comprises:
administering a non-AR focused treatment if the plurality of genomic variations includes at least one of the castration-resistant genomic variations; and
administering an AR-focused treatment if the plurality of genomic variations does not include at least one of the castration-resistant genomic variations.

20. The method of claim 19, wherein:
the non-AR focused treatment comprises one or more of immunotherapy and chemotherapy; and
the AR focused treatment comprises one or more AR-directed drugs, the AR-directed drugs comprising Abiraterone and Enzalutamide.
